SEQUENCE LISTING (81 sequences)

SEQ ID NO: 1 - DNA, 2467 bp, Burkhorderia cepacia
  CDS (258)..(761)
  CDS (764)..(2380)
  CDS (2386)..(2466)
aagctttctgtttgattgcacgcgattctaaccgagcgtctgtgaggcggaacgcgacatgcttcgtgtcgcacacgtgtcgcgccgacgacacaaaaatgcagcgaaatggctgatcgttacgaatggctgacacattgaatggactataaaaccattgtccgttccggaatgtgcgcgtacatttcaggtccgcgccgatttttgagaaatatcaagcgtggttttcccgaatccggtgttcgagagaaggaaacatgcacaacgacaacactccccactcgcgtcgccacggcgacgcagccgcatcaggcatcacgcggcgtcaatggttgcaaggcgcgctggcgctgaccgcagcgggcctcacgggttcgctgacattgcgggcgcttgcagacaaccccggcactgcgccgctcgatacgttcatgacgctttccgaatcgctgaccggcaagaaagggctcagccgcgtgatcggcgagcgcctgctgcaggcgctgcagaagggctcgttcaagacggccgacagcctgccgcagctcgccggcgcgctcgcgtccggttcgctgacgcctgaacaggaatcgctcgcactgacgatcctcgaggcctggtatctcggcatcgtcgacaacgtcgtgattacgtacgaggaagcattaatgttcggcgtcgtgtccgatacgctcgtgatccgttcgtattgccccaacaaacccggcttctgggccgacaaaccgatcgagaggcaagcctgatggccgataccgatacgcaaaaggccgacgtcgtcgtcgttggatcgggtgtcgcgggcgcgatcgtcgcgcatcagctcgcgatggcgggcaaggcggtgatcctgctcgaagcgggcccgcgcatgccgcgctgggaaatcgtcgagcgcttccgcaatcagcccgacaagatggacttcatggcgccgtacccgtcgagcccctgggcgccgcatcccgagtacggcccgccgaacgactacctgatcctgaagggcgagcacaagttcaactcgcagtacatccgcgcggtgggcggcacgacgtggcactgggccgcgtcggcgtggcgcttcattccgaacgacttcaagatgaagagcgtgtacggcgtcggccgcgactggccgatccagtacgacgatctcgagccgtactatcagcgcgcggaggaagagctcggcgtgtggggcccgggccccgaggaagatctgtactcgccgcgcaagcagccgtatccgatgccgccgctgccgttgtcgttcaacgagcagaccatcaagacggcgctgaacaactacgatccgaagttccatgtcgtgaccgagccggtcgcgcgcaacagccgcccgtacgacggccgcccgacttgttgcggcaacaacaactgcatgccgatctgcccgatcggcgcgatgtacaacggcatcgtgcacgtcgagaaggccgaacgcgccggcgcgaagctgatcgagaacgcggtcgtctacaagctcgagacgggcccggacaagcgcatcgtcgcggcgctctacaaggacaagacgggcgccgagcatcgcgtcgaaggcaagtatttcgtgctcgccgcgaacggcatcgagacgccgaagatcctgctgatgtccgcgaaccgcgatttcccgaacggtgtcgcgaacagctcggacatggtcggccgcaacctgatggaccatccgggcaccggcgtgtcgttctatgcgagcgagaagctgtggccgggccgcggcccgcaggagatgacgtcgctgatcggtttccgcgacggtccgttccgcgcgaccgaagcggcgaagaagatccacctgtcgaacctgtcgcgcatcgaccaggagacgcagaagatcttcaaggccggcaagctgatgaagcccgacgagctcgacgcgcagatccgcgaccgttccgcacgctacgtgcagttcgactgcttccacgaaatcctgccgcaacccgagaaccgcatcgtgccgagcaagacggcgaccgatgcgatcggcattccgcgccccgagatcacgtatgcgatcgacgactacgtgaagcgcggcgccgcgcatacgcgcgaggtctacgcgaccgccgcgaaggtgctcggcggcacggacgtcgtgttcaacgacgaattcgcgccgaacaatcacatcacgggctcgacgatcatgggcgccgatgcgcgcgactccgtcgtcgacaaggactgccgcacgttcgaccatccgaacctgttcatttcgagcagcgcgacgatgccgaccgtcggtaccgtaaacgtgacgctgacgatcgccgcgctcgcgctgcggatgtcggacacgctgaagaaggaagtctgaccgtgcggaaatctactctcactttcctcatcgccggctgcctcgcgttgccgggcttcgcgcgcgcggccgatgcggccgatc

SEQ ID NO: 2 - protein, 168 aa, Burkhorderia cepacia
MHNDNTPHSRRHGDAAASGITRRQWLQGALALTAAGLTGSLTLRALADNPGTAPLDTFMTLSESLTGKKGLSRVIGERLLQALQKGSFKTADSLPQLAGALASGSLTPEQESLALTILEAWYLGIVDNVVITYEEALMFGVVSDTLVIRSYCPNKPGFWADKPIERQA

SEQ ID NO: 3 - protein, 539 aa, Burkhorderia cepacia
MADTDTQKADVVVVGSGVAGAIVAHQLAMAGKAVILLEAGPRMPRWEIVERFRNQPDKMDFMAPYPSSPWAPHPEYGPPNDYLILKGEHKFNSQYIRAVGGTTWHWAASAWRFIPNDFKMKSVYGVGRDWPIQYDDLEPYYQRAEEELGVWGPGPEEDLYSPRKQPYPMPPLPLSFNEQTIKTALNNYDPKFHVVTEPVARNSRPYDGRPTCCGNNNCMPICPIGAMYNGIVHVEKAERAGAKLIENAVVYKLETGPDKRIVAALYKDKTGAEHRVEGKYFVLAANGIETPKILLMSANRDFPNGVANSSDMVGRNLMDHPGTGVSFYASEKLWPGRGPQEMTSLIGFRDGPFRATEAAKKIHLSNLSRIDQETQKIFKAGKLMKPDELDAQIRDRSARYVQFDCFHEILPQPENRIVPSKTATDAIGIPRPEITYAIDDYVKRGAAHTREVYATAAKVLGGTDVVFNDEFAPNNHITGSTIMGADARDSVVDKDCRTFDHPNLFISSSATMPTVGTVNVTLTIAALALRMSDTLKKEV

SEQ ID NO: 4 - protein, 27 aa, Burkhorderia cepacia
VRKSTLTFLIAGCLALPGFARAADAAD

SEQ ID NO: 5 - DNA, 1441 bp, Burkholderia cepacia
  CDS (121)..(1398)
tccgaacctgttcatttcgagcagcgcgacgatgccgaccgtcggtaccgtaaacgtgacgctgacgatcgccgcgctcgcgctgcggatgtcggacacgctgaagaaggaagtctgaccgtgcggaaatctactctcactttcctcatcgccggctgcctcgcgttgccgggcttcgcgcgcgcggccgatgcggccgatccggcgctggtcaagcgcggcgaatacctcgcgaccgccggcgactgcatggcctgccacaccgtgaagggcggcaagccgtacgcgggcggccttggcatgccggtaccgatgctcggcaagatctacacgagcaacatcacgcccgatcccgatacgggcatcggcaaatggacgttcgaggacttcgagcgcgcggtgcggcacggcgtgtcgaagaacggcgacaacctgtatccggcgatgccgtacgtgtcgtacgcgaagatcacggacgacgacgtacgcgcgctgtacgcctacttcatgcacggcgtcgagccggtcaagcaggcgccgccgaagaacgagattcccgcgctgctcagcatgcgctggccgctgaagatctggaactggctgttcctgaaggacggcccgtaccagccgaagccgtcgcagagcgccgaatggaatcgcggcgcgtatctggtgcagggtctcgcgcactgcagcacgtgccacacgccgcgcggcatcgcgatgcaggagaagtcgctcgacgaaaccggcggcagcttcctcgcggggtcggtgctcgccggctgggacggctacaacatcacgtcggacccgaatgcggggatcggcagctggacgcagcagcagctcgtgcagtatttgcgcaccggcagcgtgccgggcgtcgcgcaggcggccgggccgatggccgaggcggtcgagcacagcttctcgaagatgaccgaagcggacatcggtgcgatcgccacgtacgtccgcacggtgccggccgttgccgacagcaacgcgaagcagccgcggtcgtcgtggggcaagccggccgaggacgggctgaagctgcgcggtgtcgcgctcgcgtcgtcgggcatcgatccggcgcggctgtatctcggcaactgcgcgacgtgccaccagatgcagggcaagggcacgccggacggctattacccgtcgctgttccacaactccaccgtcggcgcgtcgaatccgtcgaacctcgtgcaggtgatcctgaacggcgtgcagcgcaagatcggcagcgaggatatcgggatgcccgctttccgctacgatctgaacgacgcgcagatcgccgcgctgacgaactacgtgaccgcgcagttcggcaatccggcggcgaaggtgacggagcaggacgtcgcgaagctgcgctgacatagtcgggcgcgccgacacggcgcaaccgataggacaggag

SEQ ID NO: 6 - protein, 425 aa, Burkholderia cepacia
VRKSTLTFLIAGCLALPGFARAADAADPALVKRGEYLATAGDCMACHTVKGGKPYAGGLGMPVPMLGKIYTSNITPDPDTGIGKWTFEDFERAVRHGVSKNGDNLYPAMPYVSYAKITDDDVRALYAYFMHGVEPVKQAPPKNEIPALLSMRWPLKIWNWLFLKDGPYQPKPSQSAEWNRGAYLVQGLAHCSTCHTPRGIAMQEKSLDETGGSFLAGSVLAGWDGYNITSDPNAGIGSWTQQQLVQYLRTGSVPGVAQAAGPMAEAVEHSFSKMTEADIGAIATYVRTVPAVADSNAKQPRSSWGKPAEDGLKLRGVALASSGIDPARLYLGNCATCHQMQGKGTPDGYYPSLFHNSTVGASNPSNLVQVILNGVQRKIGSEDIGMPAFRYDLNDAQIAALTNYVTAQFGNPAAKVTEQDVAKLR

SEQ ID NO: 7 - protein, 539 aa, Burkholderia cenocepacia
MADTDTQKADVVVVGSGVAGAIVAHQLAMAGKSVILLEAGPRMPRWEIVERFRNQPDKTDFMAPYPSSPWAPHPEYGPPNDYLILKGEHKFNSQYIRAVGGTTWHWAASAWRFIPNDFKMKTVYGVARDWPIQYDDLEHWYQRAEEELGVWGPGPEEDLYSPRKQAYPMPPLPLSFNEQTIKSALNGYDPKFHVVTEPVARNSRPYDGRPTCCGNNNCMPICPIGAMYNGIVHVEKAEQAGAKLIDSAVVYKLETGPDKRIVAAIYKDKTGADHRVEGKYFVLAANGIETPKILLMSANRDFPNGVANSSDMVGRNLMDHPGTGVSFYANEKLWPGRGPQEMTSLIGFRDGPFRATEAAKKIHLSNMSRINQETQKIFKAGKLMKHEELDAQIRDRSARYVQFDCFHEILPQPENRIVPSKTATDAIGIPRPEITYAIDDYVKRGAVHTREVYATAAKVLGGTDVVFNDEFAPNNHITGATIMGADARDSVVDKDCRTFDHPNLFISSSSTMPTVGTVNVTLTIAALALRMSDTLKKEV

SEQ ID NO: 8 - protein, 537 aa, Burkholderia thailandensis
MAETQQADVVVVGSGVAGAIVAHQLAMAGKSVILLEAGPRMPRWEIVERFRNQPDKMDFMAPYPSSAWAPHPEYAPPNDYLVLKGEHKFNSQYIRAVGGTTWHWAASAWRFIPNDFKMKTVYGVGRDWPIQYDDLEHFYQRAEEELGVWGPGAEEDLLSPRKAPYPMPPLPLSYNERTIKTALNNHDPKYHVVTEPVARNSRPYDGRPTCCGNNNCMPICPIGAMYNGIVHVEKAEQAGAKLIENAVVHKLEVGPQKKIVAALYKDPKGAEHRVEGKYFVLAANGIETPKLMLMSTSHDFPNGVGNSSDMVGRNLMDHPGTGVSFYASEKLWPGRGPQEMTSLIGFRDGPFRATEAAKKIHLSNLSRIDQETQKIFKAGKLLKPAELDAQIRDRSARYVQFDCFHEILPQPENRIVPSKTATDAIGIPRPEITYAIDDYVKRGAAHTREVYASAAQVLGGTDVVFNDEFAPNNHITGATIMGADPRDSVVDKDCRTFDHPNLFISSSATMPTVGTVNVTLTIAALALRISDQLKKEI

SEQ ID NO: 9 - protein, 540 aa, Ralstonia pickettii
MAQSEQTRQQADIVVVGSGVAGALVAYELARAGKSVLMLEAGPRLPRWEIVERFRNQADKMDFMAPYPSTAWAPHPEYGPPNNYLVLKGEHQFNSQYIRAVGGTTWHWAASTWRFLPNDFKLRSVYGIARDWPIQYQDLERYYGLAEEALGVWGPNDEDLGSPRSQPYPMTPLPLSFNERTIKEALNAHDASFHVVTEPVARNSRPYDGRPTCCGNNNCMPICPIGAMYNGIVHVEKAEQAGARLIENAVVFKLEVGPNKRIVAARYKDSKGAEHRVEGKWFVLAANGIETPKLMLMSTSQDFPKGVGNSSDMVGRNLMDHPGTGVSFYADRKLWPGRGPQEMTSLIGFRDGPFRATQAGKKLHLSNISRIEQETQRIFKEGKLIKPADLDARIRDQAARYVQFDSFHEILPLPENRIVPSATEVDAIGIPRPEITYHIDDYVKRSAVHTREVYATAAQVMGGTNVEFHDDFAPNNHITGATIMGADPKDSVVDKDCRTFDHPNLFISSSSTMPTVGTVNVTLTIAALALRIADQLKQEA

SEQ ID NO: 10 - protein, 540 aa, Ralstonia solanacearum
MADTRRADQADIVVVGSGVAGALVAYELARAGKSVLMLEAGPRLPRWEIVERFRNQADKMDFMAPYPSTPWAPHPEYGPSPNDYLVLKGEHKFDSQYIRAVGGTTWHWAASTWRFLPNDFKLRSVYGIARDWPLQYDDLERDYGRAEAALGVWGPNDEDLGSPRSQPYPMAPLPLSFNERTIKEALNAHDPAFHVVTEPVARNSRPYDGRPTCCGNNNCMPICPIGAMYNGIVHVEKAEQAGARLIENAVVYKLEVGAGRRIVAAHYKDPKGVDHRVEGKWFVLAANGIETPKLMLMSTSEAFPRGVGNSSDMVGRNLMDHPGTGVSFYADRKLWPGRGPQEMTSLIGFRDGPFRAMQAGKKLHLSNISRIEQETARIFKAGKLLKPAELDARIRDQAARYVQFDSFHEILPLPENRIVPSATETDALGIPRPEITYRIDDYVKRSAVHTREVYATAAKVLGATDVQFHDDFAPNNHITGATSMGADPKDSVVDKDCRTFDHPNLFISSSATMPTVGTVNVTLTIAALALRIADRLKKEA

SEQ ID NO: 11 - protein, 533 aa, Burkholderia phytofirmans
MANKNSADIVVVGSGVAGGLVAHQMALAGASVILLEAGPRIPRWQIVENFRNSPVKSDFATPYPSTPYAPHPEYAPANNYLIQKGDYPYSSQYLRLVGGTTWHWAAAAWRLLPSDFQLHKLYGVGRDWPYPYETLEPWYSAAEVQLGVSGPGNSIDLGSPRSKPYPMNPLPLSYMDQRFSDVLNAQGFKVVPEPVARNSRPYDARPTCCGNNNCMPICPIAAMYNGVVHAEKAEQAGAKLIPEAVVYRVEADNKGLITAVHYKDPNGNSTRVTGKLFVLAANGIETPKLMLMSTSDKFPHGVGNSSDQVGRNLMDHPGTGVTFLANEALWPGRGPMEMTSIVNFRDGAFRSDYAAKKLHLSNGVPTMSVTADLLKKGLTGAELDRQIRDRAARTLNINSFHEHLAEPQNRVVPSADHKDSLGIPQPEIYYSINDYVKKSAANTHELYAQIAALFGGAEVTFDDTFAPNNHIMGTTIMGSDPADSVVDADCRTHDHSNLFIASSGVMPTAASVNCTLTIAALSLKLADKLKREI

SEQ ID NO: 12 - protein, 539 aa, Burkholderia cepacia
MADTDTQKADVVVVGSGVAGAIVAHQLAMAGKSVILLEAGPRMPRWEIVERFRNQVDKTDFMAPYPSSAWAPHPEYGPPNDYLILKGEHKFNSQYIRAVGGTTWHWAASAWRFIPNDFKMKTVYGVGRDWPIQYDDIEHYYQRAEEELGVWGPGPEEDLYSPRKEPYPMPPLPLSFNEQTIKSALNGYDPKFHVVTEPVARNSRPYDGRPTCCGNNNCMPICPIGAMYNGIVHVEKAEQAGAKLIDSAVVYKLETGPDKRITAAVYKDKTGADHRVEGKYFVIAANGIETPKILLMSANRDFPNGVANSSDMVGRNLMDHPGTGVSFYANEKLWPGRGPQEMTSLIGFRDGPFRATEAAKKIHLSNMSRINQETQKIFKAGKLMKPEELDAQIRDRSARYVQFDCFHEILPQPENRIVPSKTATDAIGIPRPEITYAIDDYVKRGAVHTREVYATAAKVLGGTEVVFNDEFAPNNHITGATIMGADARDSVVDKDCRTFDHPNLFISSSSTMPTVGTVNVTLTIAALALRMSDTLKKEV

SEQ ID NO: 13 - protein, 539 aa, Burkholderia cepacia
MADTDTQKADVVVVGSGVAGAIVAHQLAMAGKSVILLEAGPRMPRWEIVERFRNQVDKTDFMAPYPSSAWAPHPEYGPPNDYLILKGEHKFNSQYIRAVGGTTWHWAASAWRFIPNDFKMKTVYGVGRDWPIQYDDIEHYYQRAEEELGVWGPGPEEDLYSPRKEPYPMPPLPLSFNEQTIKSALNGYDPKFHVVTEPVARNSRPYDGRPTCCGNNNCMPICPIGAMYNGIVHVEKAEQAGAKLIDSAVVYKLETGPDKRITAAVYKDKTGADHRVEGKYFVIAANGIETPKILLMSANRDFPNGVANSSDMVGRNLMDHPGTGVSFYANEKLWPGRGPQEMTSLIGFRDGPFRANEAAKKIHLSNMSRINQETQKIFKGGKLMKPEELDAQIRDRSARFVQFDCFHEILPQPENRIVPSKTATDAVGIPRPEITYAIDDYVKRGAVHTREVYATAAKVLGGTEVVFNDEFAPNNHITGATIMGADARDSVVDKDCRTFDHPNLFISSSSTMPTVGTVNVTLTIAALALRMSDTLKKEV

SEQ ID NO: 14 - protein, 539 aa, Burkholderia cepacia
MADTDTQKADVVVVGSGVAGAIVAHQLAMAGKSVILLEAGPRMPRWEIVERFRNQTDKTDFMAPYPSSPWAPHPEYGPPNDYLVLKGEHKFNSQYIRAVGGTTWHWAASAWRFIPNDFKMKTVYGVGRDWPIQYDDLEHYYQRAEEELGVWGPGPEEDLYSPRRQPYPMPPLPLSFNEQTIKSALNGYDPKFHVVTEPVARNSRPYDGRPTCCGNNNCMPICPIGAMYNGIVHVEKAEQAGAKLIENAVVYKLETGPNKRIVAAIYKDKSGADHRVEGKYFVVAANGIETPKILLMSANRDFPNGVANSSDMVGRNLMDHPGTGVSFYANEKLWPGRGPQEMTSLIGFRDGPFRATEAAKKIHLSNMSRINQETQKIFKGGKLMKPEELDAQIRDRSARYVQFDCFHEILPQPENRIVPSKTATDAIGIPRPEITYAIDDYVKRGAVHTCEVYATAAKVLGGTEVVFNDEFAPNNHITGATIMGADARDSVVDKDCRTFDHPNLFISSSSTMPTVGTVNVTLTIAALALRMSDTLKKEV

SEQ ID NO: 15 - protein, 539 aa, Burkholderia cepacia
MADTDTQKADIVVVGSGVAGAIVAHQLAMAGKSVILLEAGPRMPRWEIVERFRNQPDKTDFMAPYPSSPWAPHPEYGPPNDYLILKGEHKFNSQYIRAVGGTTWHWAASAWRFIPNDFKMKTVYGVARDWPIQYDDLEHWYQRAEEELGVWGPGPEEDLYSPRKQAYPMPPLPLSFNEQTIKSALNGYDPKFHVVTEPVARNSRPYDGRPTCCGNNNCMPICPIGAMYNGIVHVEKAEQAGAKLIDSAVVYKLETGPDKRIVAAIYKDKTGADHRVEGKYFVLAANGIETPKILLMSANRDFPNGVANSSDMVGRNLMDHPGTGVSFYANEKLWPGRGPQEMTSLIGFRDGPFRATEAAKKIHLSNMSRINQETQKIFKAGKLMKHEELDAQIRDRSARYVQFDCFHEILPQPENRIVPSKTATDAIGIPRPEITYAIDDYVKRGAVHTREVYATAAKVLGGTDVVFNDEFAPNNHITGATIMGADARDSVVDKDCRTFDHPNLFISSSSTMPTVGTVNVTLTIAALALRMSDTLKKEV

SEQ ID NO: 16 - protein, 539 aa, Burkholderia cepacia; identical to SEQ ID NO: 15

SEQ ID NO: 17 - DNA, 40 bp, artificial sequence
  Synthetic primer
accaccactgataaggaggtctgaccgtgcggaaatctac

SEQ ID NO: 18 - DNA, 40 bp, artificial sequence
  Synthetic primer
agcctgtgcgacttcttccttcagcgatcggtggtggtgg

SEQ ID NO: 19 - DNA, 26 bp, artificial sequence
  Synthetic primer
catgccatggcacacaacgacaacac

SEQ ID NO: 20 - DNA, 30 bp, artificial sequence
  Synthetic primer
gtcgacgatcttcttccagccgaacatcac

SEQ ID NO: 21 - DNA, 30 bp, artificial sequence
  Synthetic primer
gaattctatgcgagcgagaagctgtggccg

SEQ ID NO: 22 - DNA, 30 bp, artificial sequence
  Synthetic primer
ttcttctatgcgagcgagaagctgtggccg

SEQ ID NO: 23 - DNA, 30 bp, artificial sequence
  Synthetic primer
atcttctatgcgagcgagaagctgtggccg

SEQ ID NO: 24 - DNA, 30 bp, artificial sequence
  Synthetic primer
aaattctatgcgagcgagaagctgtggccg

SEQ ID NO: 25 - DNA, 30 bp, artificial sequence
  Synthetic primer
ctgttctatgcgagcgagaagctgtggccg

SEQ ID NO: 26 - DNA, 30 bp, artificial sequence
  Synthetic primer
aacttctatgcgagcgagaagctgtggccg

SEQ ID NO: 27 - DNA, 30 bp, artificial sequence
  Synthetic primer
cagttctatgcgagcgagaagctgtggccg

SEQ ID NO: 28 - DNA, 30 bp, artificial sequence
  Synthetic primer
cgcttctatgcgagcgagaagctgtggccg

SEQ ID NO: 29 - DNA, 30 bp, artificial sequence
  Synthetic primer
accttctatgcgagcgagaagctgtggccg

SEQ ID NO: 30 - DNA, 30 bp, artificial sequence
  Synthetic primer
gttttctatgcgagcgagaagctgtggccg

SEQ ID NO: 31 - DNA, 30 bp, artificial sequence
  Synthetic primer
tggttctatgcgagcgagaagctgtggccg

SEQ ID NO: 32 - DNA, 30 bp, artificial sequence
  Synthetic primer
tacttctatgcgagcgagaagctgtggccg

SEQ ID NO: 33 - DNA, 30 bp, artificial sequence
  Synthetic primer
cacgccggtcccgggatggtccatcaggtt

SEQ ID NO: 34 - DNA, 30 bp, artificial sequence
  Synthetic primer
cacgccggtgcccggatggtccatcaggtt

SEQ ID NO: 35 - DNA, 30 bp, artificial sequence
  Synthetic primer
gacaacctgtcgcgcatcgaccaggagacg

SEQ ID NO: 36 - DNA, 30 bp, artificial sequence
  Synthetic primer
ttcaacctgtcgcgcatcgaccaggagacg

SEQ ID NO: 37 - DNA, 30 bp, artificial sequence
  Synthetic primer
atcaacctgtcgcgcatcgaccaggagacg

SEQ ID NO: 38 - DNA, 30 bp, artificial sequence
  Synthetic primer
aaaaacctgtcgcgcatcgaccaggagacg

SEQ ID NO: 39 - DNA, 30 bp, artificial sequence
  Synthetic primer
cagaacctgtcgcgcatcgaccaggagacg

SEQ ID NO: 40 - DNA, 30 bp, artificial sequence
  Synthetic primer
cgtaacctgtcgcgcatcgaccaggagacg

SEQ ID NO: 41 - DNA, 30 bp, artificial sequence
  Synthetic primer
accaacctgtcgcgcatcgaccaggagacg

SEQ ID NO: 42 - DNA, 30 bp, artificial sequence
  Synthetic primer
tacaacctgtcgcgcatcgaccaggagacg

SEQ ID NO: 43 - DNA, 30 bp, artificial sequence
  Synthetic primer
caggtggatcttcttcgccgcttcggtcgc

SEQ ID NO: 44 - DNA, 30 bp, artificial sequence
  Synthetic primer
gcgccgaacaatcacatcacgggctcgacg

SEQ ID NO: 45 - DNA, 30 bp, artificial sequence
  Synthetic primer
gacccgaacaatcacatcacgggctcgacg

SEQ ID NO: 46 - DNA, 30 bp, artificial sequence
  Synthetic primer
gaaccgaacaatcacatcacgggctcgacg

SEQ ID NO: 47 - DNA, 30 bp, artificial sequence
  Synthetic primer
ttcccgaacaatcacatcacgggctcgacg

SEQ ID NO: 48 - DNA, 30 bp, artificial sequence
  Synthetic primer
catccgaacaatcacatcacgggctcgacg

SEQ ID NO: 49 - DNA, 30 bp, artificial sequence
  Synthetic primer
atcccgaacaatcacatcacgggctcgacg

SEQ ID NO: 50 - DNA, 30 bp, artificial sequence
  Synthetic primer
aaaccgaacaatcacatcacgggctcgacg

SEQ ID NO: 51 - DNA, 30 bp, artificial sequence
  Synthetic primer
aacccgaacaatcacatcacgggctcgacg

SEQ ID NO: 52 - DNA, 30 bp, artificial sequence
  Synthetic primer
cgtccgaacaatcacatcacgggctcgacg

SEQ ID NO: 53 - DNA, 30 bp, artificial sequence
  Synthetic primer
agtccgaacaatcacatcacgggctcgacg

SEQ ID NO: 54 - DNA, 30 bp, artificial sequence
  Synthetic primer
actccgaacaatcacatcacgggctcgacg

SEQ ID NO: 55 - DNA, 30 bp, artificial sequence
  Synthetic primer
tacccgaacaatcacatcacgggctcgacg

SEQ ID NO: 56 - DNA, 30 bp, artificial sequence
  Synthetic primer
gaattcgtcgttgaacacgacgtccgtgcc

SEQ ID NO: 57 - DNA, 30 bp, artificial sequence
  Synthetic primer
gaactcgtcgttgaacacgacgtccgtgcc

SEQ ID NO: 58 - DNA, 31 bp, Artificial Sequence
  Synthetic primer
gggcaccggcgtgcagttctatgcgaacgag

SEQ ID NO: 59 - DNA, 37 bp, Artificial Sequence
  Synthetic primer
gaagaagatccacctgtacaacatgtcgcgcatcaac

SEQ ID NO: 60 - DNA, 44 bp, Artificial Sequence
  Synthetic primer
gtcgtgttcaacgacgaattctacccgaacaatcacatcacggg

SEQ ID NO: 61 - DNA, 31 bp, Artificial Sequence
  Synthetic primer
ctcgttcgcatagaactgcacgccggtgccc

SEQ ID NO: 62 - DNA, 37 bp, Artificial Sequence
  Synthetic primer
gttgatgcgcgacatgttgtacaggtggatcttcttc

SEQ ID NO: 63 - DNA, 44 bp, Artificial Sequence
  Synthetic primer
cccgtgatgtgattgttcgggtagaattcgtcgttgaacacgac

SEQ ID NO: 64 - DNA, 31 bp, Artificial Sequence
  Synthetic primer
gggcaccggcgtgcagttctatgcgagcgag

SEQ ID NO: 65 - DNA, 37 bp, Artificial Sequence
  Synthetic primer
gaagaagatccacctgtacaacctgtcgcgcatcgac

SEQ ID NO: 66 - DNA, 31 bp, Artificial Sequence
  Synthetic primer
ctcgctcgcatagaactgcacgccggtgccc

SEQ ID NO: 67 - DNA, 37 bp, Artificial Sequence
  Synthetic primer
gtcgatgcgcgacaggttgtacaggtggatcttcttc

SEQ ID NO: 68 - DNA, 31 bp, Artificial Sequence
  Synthetic primer
gggcaccggcgtgcagttctatgcggaccgc

SEQ ID NO: 69 - DNA, 37 bp, Artificial Sequence
  Synthetic primer
caagaagctgcacctgtacaacatctcgcgcatcgag

SEQ ID NO: 70 - DNA, 44 bp, Artificial Sequence
  Synthetic primer
gtcgagttccacgacgacttctacccgaacaatcacatcacggg

SEQ ID NO: 71 - DNA, 31 bp, Artificial Sequence
  Synthetic primer
gcggtccgcatagaactgcacgccggtgccc

SEQ ID NO: 72 - DNA, 37 bp, Artificial Sequence
  Synthetic primer
ctcgatgcgcgagatgttgtacaggtgcagcttcttg

SEQ ID NO: 73 - DNA, 44 bp, Artificial Sequence
  Synthetic primer
cccgtgatgtgattgttcgggtagaagtcgtcgtggaactcgac

SEQ ID NO: 74 - DNA, 44 bp, Artificial Sequence
  Synthetic primer
gtccagttccacgacgacttctacccgaacaatcacatcacggg

SEQ ID NO: 75 - DNA, 44 bp, Artificial Sequence
  Synthetic primer
cccgtgatgtgattgttcgggtagaagtcgtcgtggaactggac

SEQ ID NO: 76 - DNA, 31 bp, Artificial Sequence
  Synthetic primer
gggcaccggcgtgcagttcctggcgaacgag

SEQ ID NO: 77 - DNA, 37 bp, Artificial Sequence
  Synthetic primer
gaagaagctgcacctgtacaacggcgtcccgacgatg

SEQ ID NO: 78 - DNA, 44 bp, Artificial Sequence
  Synthetic primer
gtcacgttcgacgacacgttctacccgaacaatcacatcatggg

SEQ ID NO: 79 - DNA, 31 bp, Artificial Sequence
  Synthetic primer
ctcgttcgccaggaactgcacgccggtgccc

SEQ ID NO: 80 - DNA, 37 bp, Artificial Sequence
  Synthetic primer
catcgtcgggacgccgttgtacaggtgcagcttcttc

SEQ ID NO: 81 - DNA, 44 bp, Artificial Sequence
  Synthetic primer
cccatgatgtgattgttcgggtagaacgtgtcgtcgaacgtgac

(19) United States
(12) Patent Application Publication
(10) Pub. No.: US 2012/0107903 A1
(43) Pub. Date: May 3, 2012

(54) Mutant Glucose Dehydrogenase

(75) Inventors: Koji Sode, Tokyo (JP); Katsuhiro Kojima, Tokyo (JP)

(73) Assignees: BIOENGINEERING LABORATORIES, LLC, Tokyo (JP); ARKRAY, Inc., Kyoto (JP)

(21) Appl. No.: 13/282,243

(22) Filed: Oct. 26, 2011

(30) Foreign Application Priority Data
Oct. 27, 2010 (JP) JP 2010-240426

(51) Int. Cl. C12N 9/04; C12N 1/15; C12N 1/21; C12N 1/19; C12N 9/96; C12N 15/53

(52) U.S. Cl. 435/188; 435/190; 536/23.2; 435/252.31; 435/254.21; 435/254.3

(57) ABSTRACT

A mutant glucose dehydrogenase having an amino acid sequence at least 80% identical to SEQ ID NO:3 and having glucose dehydrogenase activity, wherein amino acid residues corresponding to positions 326, 365 and 472 of said amino acid sequence are replaced with glutamine, tyrosine and tyrosine, respectively, and wherein said mutant glucose dehydrogenase shows an improved substrate specificity to glucose and a reduced reactivity to disaccharides.

### TECHNICAL FIELD

The present invention relates to a mutant glucose dehydrogenase showing an improved substrate specificity. Specifically, the present invention relates to a glucose dehydrogenase comprising a mutant-type α-subunit which is produced by introducing mutations into amino acid residues of its α-subunit that constitutes a cytochrome C-containing glucose dehydrogenase (hereinafter also referred to as CyGDH), and relates to a gene thereof. The glucose dehydrogenases of the present invention can be suitably used for glucose sensors, glucose assay kits and so forth, and are useful in the fields of biochemistry, clinical medicine and so forth.

### BACKGROUND ART

At present, a wild-type CyGDH, a PQQGDH using pyrroloquinoline quinone as a coenzyme, or the like is used for self-monitoring blood glucose sensors. The wild-type CyGDH and PQQGDH have a drawback in that they are incapable of accurately measuring the blood sugar level in the case where the blood maltose level of patients is high, because they react not only with glucose but also with maltose. Especially in Japan and the United Kingdom, maltose is used as an energy material in infusion solutions, and, in fact, there have been some accidents caused by the wrong measurement results wherein patients who received administration of such infusion solution by peritoneal dialysis or the like and whose blood sugar level was low were mistaken for high blood sugar level due to a sensor that uses a PQQGDH for measuring the blood sugar level.

In wild-type CyGDHs, the reactivities to maltose are high as compared to those to glucose, and therefore, even if the glucose concentration is 50 mg/dL, the measurement results of the blood sugar level in the case where the maltose concentration is 100 mg/dL, will indicate a value higher by 170%.

In view of these circumstances, a mutant enzyme of CyGDH (a mutant glucose dehydrogenase that is obtained by introducing mutations into 326th and 365th amino acid residues of its α-subunit so that the 326th and 365th amino acid residues are replaced with a glutamine (Q) and a tyrosine (Y), respectively) (hereinafter referred to as CyGDH (QY) or also referred to as simply QY or QYA) has been devised (WO2006/137283). According to this invention, the apparent increase of the blood sugar level can be suppressed so that, when the glucose concentration is 50 mg/dL, the measurement results of the blood sugar level in the case where the maltose concentration is 100 mg/dL will indicate a value higher by up to 36%. However, the effect to avoid the influence of maltose was not perfect.

### SUMMARY OF THE INVENTION

An object of the present invention is to provide a CyGDH showing improved substrate specificity to glucose as compared to the CyGDH (QY).

The present inventors intensively studied for attaining the above-described object to discover that, as compared to the CyGDH (QY), the substrate specificities are further improved by replacing the sites corresponding to positions 326 and 365 of amino acid residues of its α-subunit constituting the CyGDH with glutamine and tyrosine, respectively, and further replacing the site corresponding to position 472 with tyrosine, and that, also in GDH homologues, similar effect can be obtained by replacing the sites corresponding to positions 326, 365 and 472 with glutamine, tyrosine and tyrosine, respectively, thereby completing the present invention.

That is to say, the present invention is as follows.

(1) A mutant glucose dehydrogenase having an amino acid sequence at least 80% identical to SEQ ID NO:3 and having glucose dehydrogenase activity,

wherein amino acid residues corresponding to positions 326, 365 and 472 of said amino acid sequence are replaced with glutamine, tyrosine and tyrosine, respectively, and

wherein said mutant glucose dehydrogenase shows an improved substrate specificity to glucose and a reduced reactivity to disaccharides.

(2) The mutant glucose dehydrogenase according to (1), which has an amino acid sequence at least 90% identical to SEQ ID NO:3.
(3) The mutant glucose dehydrogenase according to (1), which has the amino acid sequence of SEQ ID NO:3 except for the positions corresponding to positions 326, 365 and 472.
(4) The mutant glucose dehydrogenase according to (1), which has the amino acid sequence of SEQ ID NO:7 except for the positions corresponding to positions 326, 365 and 472.
(5) The mutant glucose dehydrogenase according to (I), which has the amino acid sequence of SEQ ID NO:8 except for the positions corresponding to positions 326, 365 and 472.
(6) The mutant glucose dehydrogenase according to (I), which has the amino acid sequence of SEQ ID NO:9 except for the positions corresponding to positions 326, 365 and 472.
(7) The mutant glucose dehydrogenase according to (1), which has the amino acid sequence of SEQ NO:10 except for the positions corresponding to positions 326, 365 and 472.
(8) The mutant glucose dehydrogenase according to (1), wherein said mutant glucose dehydrogenase shows an improved substrate specificity to glucose and a reduced reactivity to disaccharides as compared to a glucose dehydrogenase whose amino acid residues corresponding to positions 326 and 365 in said amino acid sequence are replaced with glutamine and tyrosine, respectively, but whose amino acid residue corresponding to position 472 is not replaced.
(9) The mutant glucose dehydrogenase according to (1), wherein the disaccharides are maltose.
(10) A mutant glucose dehydrogenase complex comprising at least the mutant glucose dehydrogenase according to (1) and an electron transfer subunit.
(11) The glucose dehydrogenase complex according to (10), wherein the electron transfer subunit is cytochrome C.
(12) A DNA coding for the mutant glucose dehydrogenase according to (1).
(13) A microorganism having the DNA according to (12) and producing the mutant glucose dehydrogenase according to (1).
(14) A microorganism having the DNA according to (12) and producing the mutant glucose dehydrogenase complex according to (10).
(15) A glucose assay kit comprising the mutant glucose dehydrogenase according to (1).
(16) A glucose assay kit comprising the mutant glucose dehydrogenase complex according to (10).
(17) A glucose assay kit comprising the microorganism according to (13).
(18) A glucose sensor comprising the mutant glucose dehydrogenase according to (1).
(19) A glucose sensor comprising the mutant glucose dehydrogenase complex according to (10).
(20) A glucose sensor comprising the microorganism according to (13).

### BRIEF DESCRIPTION OF THE DRAWINGS

FIG. 1 shows the structure of a glucose sensor.

FIG. 2 shows reagent parts of a glucose sensor.

FIG. 3 shows the influence of the maltose concentrations (100 mg/dL, 200 mg/dL and 300 mg/dL) on the blood sugar level when the blood sugar levels in the case where the glucose concentration was 50 mg/dL were measured using a colorimetric sensor.

FIG. 4 shows the influence of the maltose concentrations (100 mg/dL, 200 mg/dL and 300 mg/dL) on the blood sugar level when the blood sugar levels in the case where the glucose concentration was 50 mg/dL, were measured using an electrode sensor.

FIG. 5 shows the structure of an electrode glucose sensor.

### MODE FOR CARRYING OUT THE INVENTION

Hereinafter, the present invention will be described in detail. The mutant GDHs of the present invention can be produced by introducing specific mutations into an α-subunit of a wild-type GDH. Examples of the wild-type GDH include a GDH produced by Burkholderia cepacia. Examples of the GDH of Burkholderia cepacia include GDHs produced by Burkholderia cepacia KS1, JCM2800 and JCM2801 strain. The KS1 strain has been deposited in the International Patent Organism Depositary, National Institute of Advanced Industrial Science and Technology (Tsukuba Central 6, 1-1-1 Higashi, Tsukuba, Ibaraki 305-8566, Japan) on Sep. 25, 2000, under Accession No. FERM BP-7306.

The nucleotide sequence of a chromosomal DNA fragment that contains a GDH α-subunit gene and a part of a GDH β-subunit gene of the KS1 strain is shown in SEQ ID NO:1 (U.S. Published Patent Application No. 2004/0023330). There are three open reading frames (ORFs) in this nucleotide sequence, and the second and third ORFs from the 5′-terminal of the sequence encode an α-subunit (SEQ ID NO:3) and β-subunit (SEQ ID NO:4), respectively. The first ORF from the 5′-terminal of the sequence is presumed to code for a γ-subunit (SEQ ID NO:2). Further, the nucleotide sequence of a fragment that contains a full-length β-subunit gene is shown in SEQ ID NO:5. Furthermore, the amino acid sequence of the β-subunit is shown in SEQ ID NO:6 (EP1498484A). The sequence of the amino acid Nos. 1 to 22 in SEQ ID NO:6 is presumed to be a signal peptide.

Additionally, besides these, each α-subunit of a putative oxidoreductase of Burkholderia cenocepacia J2315 strain (SEQ ID NO:7), a hypothetical protein BthaT_{—}07876 of Burkholderia thailandensis TXDOH strain (SEQ ID NO:8), a FAD dependent oxidoreductase of Ralstonia pickettii 12D strain (SEQ ID NO:9), a transmembrane dehydrogenase of Ralstonia solanacearum IPO1609 strain (SEQ ID NO:10) and a glucose-methanol-choline oxidoreductase of Burkholderia phytofirmans PsJN strain (SEQ ID NO:11), which are homologues of the GDH of Burkholderia cepacia KS1 strain, can be also used in the same manner as the GDH of Burkholderia cepacia KS1 strain.

All of the amino acid sequences as shown in SEQ ID NOs:7 to 11 have been registered in the NCBI (National Center for Biotechnology Information, the U.S.) database. The sequence of SEQ ID NO:7 has been registered under Accession No. YP_{—}002234347; the sequence of SEQ ID NO:8 has been registered under Accession No. ZP_{—}02370914; the sequence of SEQ ID NO:9 has been registered under Accession No. YP_{—}002980762; the sequence of SEQ ID NO:10 has been registered under Accession No. YP_{—}002260434; and the sequence of SEQ ID NO:11 has been registered under Accession No. YP_{—}001890482.

The Burkholderia cenocepacia J2315 strain has been deposited as LMG 16656, ATCC BAA-245, CCM 4899, CCUG 48434 and NCTC 13227. The Burkholderia phytofirmans PsJN strain has been deposited as LMG 22487 and CCUG 49060.

In addition, each GDH α-subunit derived from other Burkholderia cepacia strains whose genus is the same as the Burkholderia cepacia KS1 strain, such as JCM2800, JCM2801, JCM5506, JCM5507 and IF014595 (SEQ ID NOs:12 to 16), can be also used in the same manner as the GDH of Burkholderia cepacia KS1 strain. The JCM2800, JCM2801, JCM5506 and JCM5507 have been preserved in Japan Collection of Microorganisms (JCM), RIKEN. The IF014595 has been preserved in Institute for Fermentation, Osaka (IFO).

The mutant GDHs of the present invention may be an α-subunit alone, a complex of an α-subunit and a β-subunit, a complex of an α-subunit and a γ-subunit, or a complex consisting of an α-subunit, a β-subunit and a γ-subunit. In the present specification, a GDH complex containing a β-subunit is referred to as a CyGDH, and a GDH complex not containing any β-subunit is referred to as a GDH. All of the mutant GDHs of the present invention are mutants wherein specific mutations (mutations at positions 326, 365 and 472 or mutations at positions corresponding to these) are introduced into their α-subunits. However, the mutant GDHs of the present invention may have a conservative mutation(s) in addition to such specific mutations. In addition, other subunits may be a wild type and/or may have a conservative mutation(s). The term “a conservative mutation(s)” means a mutation(s) that does(do) not substantially affect GDH activity.

The mutant-type α-subunits of the present invention preferably have an amino acid sequence as shown in any one of SEQ ID NOs:3 and 7 to 11 except for the above-mentioned specific mutations. In addition, the mutant-type α-subunits may have a conservative mutation(s) as described above, as long as they have GDH activity. That is to say, they may be proteins which have an amino acid sequence including a substitution(s), a deletion(s), an insertion(s) and/or an addition(s) of one or more amino acid residues in the amino acid sequences of SEQ ID NOs:3 and 7 to 11 in addition to the above-described specific mutations. Although an amino acid sequence that can be coded for by the nucleotide sequence of SEQ ID NO:1 is shown in SEQ ID NO:3, the methionine residue of the N-terminus may be eliminated after translation. The term “one or more” as described above means preferably 1 to 10, more preferably 1 to 5, especially preferably 1 to 3. Further, the mutant-type α-subunits of the present invention have an amino acid identity of at least 80%, preferably 85%, more preferably 90%, to the amino acid sequence shown in SEQ ID NO:3.

In addition, the β-subunits typically have the amino acid sequence of SEQ ID NO:6. However, as long as they can function as a β-subunit of a CyGDH, they may be proteins which have an amino acid sequence including a substitution(s), a deletion(s), an insertion(s) and/or an addition(s) of one or more amino acid residues in the amino acid sequence consisting of the amino acid Nos. 23 to 425 of SEQ ID NO:6. In addition, as long as they can function as a β-subunit of a CyGDH, they may be β-subunits of strains other than KS1 strain, and may be proteins which have an amino acid sequence including a substitution(s), a deletion(s), an insertion(s) and/or an addition(s) of one or more amino acid residues in the amino acid sequences of the said β-subunits of strains other than KS1 strain. The term “one or more” as described above means preferably 1 to 20, more preferably 1 to 10, especially preferably 1 to 5.

And, the wording “function as a β-subunit of a CyGDH” means that, when the β-subunit forms a complex together with an α-subunit, the β-subunit functions as an electron transfer subunit, namely, cytochrome C, without adversely affecting GDH activity of the said complex.

Specific examples of the wild-type α-subunit gene include a DNA which contains a nucleotide sequence consisting of the nucleotide Nos. 764 to 2380 of SEQ ID NO:1. Additionally, the α-subunit gene may be a DNA which has a nucleotide sequence consisting of the nucleotide Nos. 764 to 2380 of the nucleotide sequence of SEQ ID NO:1, or may be a DNA which hybridizes under stringent conditions with a probe that can be prepared from said nucleotide sequence consisting of the nucleotide Nos. 764 to 2380 of the nucleotide sequence of SEQ ID NO:1, and which codes for a protein that has GDH activity.

Specific examples of the β-subunit gene include a DNA which contains a nucleotide sequence consisting of the nucleotide Nos. 187 to 1398 of SEQ ID NO:5. Additionally, the β-subunit gene may be a DNA which has a nucleotide sequence consisting of the nucleotide Nos. 187 to 1398 of SEQ ID NO:5, or may be a DNA which hybridizes under stringent conditions with a probe that can be prepared from said nucleotide sequence consisting of the nucleotide Nos. 187 to 1398 of SEQ ID NO:5, and which codes for a protein that can function as β-subunit.

Examples of the stringent conditions as mentioned above include conditions wherein DNAs having a homology of preferably 80%, more preferably 90% or more, especially preferably 95% or more, hybridize with each other, and more specifically, conditions of 0.1×SSC, 0.1% SDS and 60° C.

The α- and β-subunit genes can be obtained by PCR using chromosomal DNA of Burkholderia cepacia KS1 strain as a template, for example. Primers for the PCR can be prepared by chemically synthesizing on the basis of the above-described nucleotide sequence. Alternatively, they can also be obtained from the chromosomal DNA of Burkholderia cepacia KS1 strain by hybridization wherein oligonucleotides made on the basis of the above-described sequence are used as probes. Also, Burkholderia cenocepacia J2315 strain, Burkholderia thailandensis TXDOH strain, Ralstonia pickettii 12D strain, Ralstonia solanacearum IPO1609 strain and Burkholderia phytofirmans PsJN strain other than KS1 strain may be used.

The mutant GDHs of the present invention are mutants obtained by introduction of the specific mutations into the wild-type GDHs or the GDHs having a conservative mutation(s) as described above, and, as a result of this, they show improved substrate specificities to glucose. The wording “show an improved substrate specificity to glucose” includes showing a reduced reactivity to other sugars such as monosaccharides, disaccharides, oligosaccharide or the like, for example, maltose, galactose, xylose or the like, while substantially maintaining the reactivity to glucose, or showing an improved reactivity to glucose as compared to the reactivity to other sugars. For example, even if the reactivity to glucose is reduced, the substrate specificity to glucose is improved when the reactivity to other sugars is reduced more than that. And, even if the reactivity to other sugars is increased, a substrate specificity to glucose is improved when the substrate specificity to glucose is increased more than that. Specifically, for example, if the improvement of the substrate specificity of a mutant-type enzyme relative to a wild-type enzyme (a ratio of a specific activity to glucose and a specific activity to other sugars such as maltose) (this improvement is represented by the following formula) is 10% or more, preferably 20% or more, more preferably 40% or more, then the substrate specificity to glucose is improved. For example, if the substrate specificity in a wild-type enzyme is 60% and the substrate specificity in a mutant-type GDH is 40%, then the substrate specificity to glucose is improved by 33%.

Substrate Specificity=(specific activity to sugars other than glucose/specific activity to glucose)×100

Improvement of Substrate Specificity=(A−B)×100/A

A: Substrate specificity of wild-type enzyme

B: Substrate specificity of mutant-type enzyme

In a mutant-type GDH, the reactivity to maltose (specific activity) is 1% or less, preferably 0.5% or less, of the reactivity to glucose (specific activity).

The mutant-type GDHs of the present invention preferably show improved substrate specificities to glucose and show reduced reactivities to disaccharides as compared to those of a glucose dehydrogenase whose amino acid residues corresponding to positions 326 and 365 of the amino acid sequence of SEQ ID NO:3 are replaced with glutamine and tyrosine, respectively, but whose amino acid residue corresponding to position 472 is not replaced.

The “mutations” in the present invention are specifically as follows.

(1) Substitution of serine residue corresponding to position 326 of the amino acid sequence of SEQ ID NO:3 to glutamine.

(2) Substitution of serine residue corresponding to position 365 of the amino acid sequence of SEQ ID NO:3 to tyrosine.

(3) Substitution of alanine residue corresponding to position 472 of the amino acid sequence of SEQ ID NO:3 to tyrosine.

The positions of the amino acid substitution mutations as described above are positions in SEQ ID NO:3, i.e., the amino acid sequence of a wild-type GDH α-subunit of Burkholderia cepacia KS1 strain. However, in the cases of homologues or variants of the GDH α-subunit which have an amino acid sequence including a substitution(s), a deletion(s), an insertion(s) and/or an addition(s) of one or more amino acid residues in the amino acid sequence of SEQ ID NO:3 in addition to the above-described specific mutations, the positions as described above mean positions corresponding to the above-described amino acid substitution positions in the amino acid sequence alignment of the homologue or variant with SEQ ID NO:3. For example, in the case of a conservative variant of the GDH α-subunit which has a deletion of one amino acid residue in the region from position 1 to position 364, the position 365 as described above means position 364 of this variant.

In SEQ ID NO:7, residues corresponding to positions 326, 365 and 472 of SEQ ID NO:3 are residues of the same positions 326, 365 and 472, respectively.

In SEQ ID NO:8, residues corresponding to positions 326, 365 and 472 of SEQ ID NO:3 are residues of positions 324, 363 and 470, respectively.

In SEQ ID NO:9, residues corresponding to positions 326, 365 and 472 of SEQ ID NO:3 are residues of positions 327, 366 and 473, respectively.

In SEQ ID NO:10, residues corresponding to positions 326, 365 and 472 of SEQ ID NO:3 are residues of positions 327, 366 and 473, respectively.

In SEQ ID NO:11, residues corresponding to positions 326, 365 and 472 of SEQ ID NO:3 are residues of positions 322, 361 and 466, respectively.

In addition, amino acid sequence identities of the amino acid sequence shown in SEQ ID NO:3 to SEQ ID NOs:7 to 11 are 96%, 93%, 82%, 82%, 63%, respectively.

Amino acid sequence alignment of SEQ ID NOs:3 and 7 to 11 is shown in Table 1 below.

TABLE 1

| | | |
|---|---|---|
| SEQ ID NO: 3 | 1 MADTDT--QKADVVVVGSGVAGAIVAHQLAMAGKAVILLEAGPRMPRWEI | 48 |
| SEQ ID NO: 7 | 1 MADTDT--QKADVVVVGSGVAGAIVAHQLAMAGKSVILLEAGPRMPRWEI | 48 |
| SEQ ID NO: 8 | 1 MAET----QQADVVVVGSGVAGAIVAHQLAMAGKSVILLEAGPRMPRWEI | 46 |
| SEQ ID NO: 9 | 1 MAQSEQTRQQADIVVVGSGVAGALVAYELARAGKSVLMLEAGPRLPRWEI | 50 |
| SEQ ID NO: 10 | 1 MADTRR-ADQADIVVVGSGVAGALVAYELARAGKSVLMLEAGPRLPRWEI | 49 |
| SEQ ID NO: 11 | 1 MANKNS----ADIVVVGSGVAGGLVAHQMALAGASVILLEAGPRIPRWQI | 46 |
| SEQ ID NO: 3 | 49 VERFRNQPDKMDFMAPYPSSPWAPHPEYGP-PNDYLILKGEHKFNSQYIR | 97 |
| SEQ ID NO: 7 | 49 VERFRNQPDKTDFMAPYPSSPWAPHPEYGP-PNDYLILKGEHKFNSQYIR | 97 |
| SEQ ID NO: 8 | 47 VERFRNQPDKMDFMAPYPSSAWAPHPEYAP-PNDYLVLKGEHKFNSQYIR | 95 |
| SEQ ID NO: 9 | 51 VERFRNQADKMDFMAPYPSTAWAPHPEYGP-PNNYLVLKGEHQFNSQYIR | 99 |
| SEQ ID NO: 10 | 50 VERFRNQADKMDFMAPYPSTPWAPHPEYGPSPNDYLVLKGEHFDKSQYIR | 99 |
| SEQ ID NO: 11 | 47 VENFRNSPVKSDFATPYPSTPYAPHPEYAP-ANNYLIQKGDYPYSSQYLR | 95 |
| SEQ ID NO: 3 | 98 AVGGTTWHWAASAWRFIPNDFKMKSVYGVGRDWPIQYDDLEPYYQRAEEE | 147 |
| SEQ ID NO: 7 | 98 AVGGTTWHWAASAWRFIPNDFKMKTVYGVARDWPIQYDDLEHWYQRAEEE | 147 |
| SEQ ID NO: 8 | 96 AVGGTTWHWAASAWRFIPNDFKMKTVYGVGRDWPIQYDDLEHFYQRAEEE | 145 |
| SEQ ID NO: 9 | 100 AVGGTTWHWAASTWRFLPNDFKLRSVYGIARDWPIQYDDLERYYGLAEEA | 149 |
| SEQ ID NO: 10 | 100 AVGGTTWHWAASTWRFLPNDFKLRSVYGIARDWPLQYDDLERDYGRAEAA | 149 |
| SEQ ID NO: 11 | 96 LVGGTTWHWAAAAWRLLPSDFQLHKLYGVGRDWPYPYFTLEPWYSAAEVQ | 145 |
| SEQ ID NO: 3 | 148 LGVWGPGPEEDLYSPRKQPYPMPPLPLSFNEQTIKTALNNYDPKFHVVTE | 197 |
| SEQ ID NO: 7 | 148 LGVWGPGPEEDLYSPRKQAYPMPPLPLSFNEGTIKSALNGYDPKFHVVTE | 197 |
| SEQ ID NO: 8 | 146 LGVWGPGAEEDLLSPRKAPYPMPPLPLSYNERTIKTALNNHDPKYHVVTE | 195 |
| SEQ ID NO: 9 | 150 LGVWGPN-DEDLGSPRSQPYPMTPLPLSFNERTIKEALNAHDASFHVVTE | 198 |
| SEQ ID NO: 10 | 150 LGVWGPN-DEDLGSPRSQPYPMAPLPLSFNERTIKEALNAHDPAFHVVTE | 198 |
| SEQ ID NO: 11 | 146 LGVSGPGNSIDLGSPRSKPYPMNPLPLSYMDQRFSDVLNAQG--FKVVPE | 193 |
| SEQ ID NO: 3 | 198 PVARNSRPYDGRPTCCGNNNCMPICPIGAMYNGIVHVEKAERAGAKLIEN | 247 |
| SEQ ID NO: 7 | 198 PVARNSRPYDGRPTCCGNNNCMPICPIGAMYNGIVHVEKAEQAGAKLIDS | 247 |
| SEQ ID NO: 8 | 196 PVARNSRPYDGRPTCCGNNWCMPICPIGAMYNGIVHVEKAEQAGAKLIEN | 245 |
| SEQ ID NO: 9 | 199 PVARNSRPYDGRPTCCGNNNCMPICPIGAMYNGIVHVEKAEQAGARLIEN | 248 |
| SEQ ID NO: 10 | 199 PVARNSRPYDGRPTCCGNNNCMPICPIGAMYNGIVHVEKAEQAGARLIEN | 248 |
| SEQ ID NO: 11 | 194 PVARNSRPYDARPTCCGNNNCMPICPIAAMYNGVVHAEKAEQAGARLIPE | 243 |
| SEQ ID NO: 3 | 248 AVVYKLETGPDKRIVAALYKDKTGAEHRVEGKYFVLAANGIETPKILLMS | 297 |
| SEQ ID NO: 7 | 248 AVVYKLETGPDKRIVAAIYKDKTGADHRVEGKYFVLAANGIETPKILLMS | 297 |
| SEQ ID NO: 8 | 246 AVVHKLEVGPQKKIVAALYKDPKGAEHRVEGKYFVLAANGIETPKLMLMS | 295 |
| SEQ ID NO: 9 | 249 AVVFKLEVGPNKRIVAARYKDSKGAEHRVEGKWFVLAANGIETPKLMLMS | 298 |
| SEQ ID NO: 10 | 249 AVVYKLEVGAGRRIVAAHYKDPKGVDHRVEGKWFVLAANGIETPKLMLMS | 298 |
| SEQ ID NO: 11 | 242 AVVYRVEADNKGLITAVHYKDPNGNSTRVTGKLFVLAANGIETPKLMLMS | 293 |
| SEQ ID NO: 3 | 298 ANRDFPNGVANSSDMVGRNLMDHPGTGVSFYASEKLWPGRGPQEMTSLIG | 347 |
| SEQ ID NO: 7 | 298 ANRDFPNGVANSSDMVGRNLMDHPGTGVSFYANEKLWPGRGPQEMTSLIG | 347 |
| SEQ ID NO: 8 | 296 TSHDFPNGVGNSSDMVGRNLMDHPGTGVSFYASEKLWPGRGPQEMTSLIG | 345 |
| SEQ ID NO: 9 | 299 TSQDFPKGVGNSSDMVGRNLMDHPGTGVSFYADRKLWPGRGPQEMTSLIG | 348 |
| SEQ ID NO: 10 | 799 TSEAFPRGVGNSSDMVGRNLMDHPGTGVSFYADRKLWPGRGPQEMTSLIG | 348 |
| SEQ ID NO: 11 | 294 TSDKFPHGVGNSSDQVGRNLNDHPGTGVTFLANEALWPGRGPMEMTSIVN | 343 |
| SEQ ID NO: 3 | 348 FRDGPFRATEAAKKIHLSNLSRIDQETQKIFKAGKLMKPDELDAQIRDRS | 397 |
| SEQ ID NO: 7 | 348 FRDGPFRATEAAKKIHLSNMSRINQETQKIFKAGKLMKHEELDAQIRDRS | 397 |
| SEQ ID NO: 8 | 346 FRDGPFRATEAAKKIHLSNLSRIDQETQKIFKAGKLLKPAELDAQIRDRS | 395 |
| SEQ ID NO: 9 | 349 FRDGPFRATQAGKKLHLSNISRIEQETQRIFKEGKLIKPADLDARIRDQA | 398 |
| SEQ ID NO: 10 | 349 FRDGPFRAMQAGKKLHLSNISRIEQETARIFKAGKLLKPAELDARIRDQA | 398 |
| SEQ ID NO: 11 | 344 FRDGAFRSDYAAKKLHLSNGVPTMSVTADLLKKG--LTGAELDRQIRDRA | 391 |
| SEQ ID NO: 3 | 398 ARYVQFDCFHEILPQPENRIVPSKTATDAIGIPRPEITYAIDDYVKRGAA | 447 |
| SEQ ID NO: 7 | 398 ARYVQFDCFHEILPQPENRIVPSKTATDAIGIPRPEITYAIDDYVKRGAV | 447 |
| SEQ ID NO: 8 | 396 ARYVQFDCFHEILPQPENRIVPSKTATDAIGIPRPEITYAIDDYVKRGAA | 445 |
| SEQ ID NO: 9 | 399 ARYVQFDSFHEILPLPENRIVPSATEVDAIGIPRPEITYHIDDYVKRSAV | 448 |
| SEQ ID NO: 10 | 399 ARYVQFDSFHEILPLPENRIVPSATETDALGIPRPEITYRIDDYVKRSAV | 448 |
| SEQ ID NO: 11 | 392 ARTLNINSFHEHLAEPQNRVVPSADHKDSLGIPQPEIYYSINDYVKKSAA | 441 |
| SEQ ID NO: 3 | 448 HTREVYATAAKVLGGTDVVFNDEFAPNNHITGSTIMGADARDSVVDKDCR | 497 |
| SEQ ID NO: 7 | 448 HTREVYATAAKVLGGTDVVFNDEFAPNNHITGATIMGADARDSVVDKDCR | 497 |
| SEQ ID NO: 8 | 446 HTREVYASAAQVLGGTDVVFNDEFAPNNHITGATIMGADPRDSVVDKDCR | 495 |
| SEQ ID NO: 9 | 449 HTREVYATAAQVMGGTNVEFHDDFAPNNHITGATIMGADPKDSVVDKDCR | 498 |
| SEQ ID NO: 10 | 449 HTREVYATAAKVLGATDVQFHDDFAPNNHITGATIMGADPKDSVVDKDCR | 498 |
| SEQ ID NO: 11 | 442 NTHELYAQIAALFGGAEVTFDDTFAPNNHIMGTTIMGSDPADSVVDADCR | 491 |
| SEQ ID NO: 3 | 498 TEDHPNLFISSSATMPTVGTVNVTLTIAALALRMSDTLKKEV | 539 |
| SEQ ID NO: 7 | 498 TFDHPNLFISSSSTMPTVGTVNVTLTIAALALRMSDTLKKEV | 539 |
| SEQ ID NO: 8 | 496 TEDHPNITISSSAIMPTVGTVNVTLTIARLALRISDQLKKEI | 537 |
| SEQ ID NO: 9 | 499 TFDHPNLFISSSSTMPTVGTVNVTLTIAALALRIADQLKQEA | 540 |
| SEQ ID NO: 10 | 499 TFDHPNLFISSSATMPTVGTVNVTLTIAALALRIADRLKKEA | 540 |
| SEQ ID NO: 11 | 492 THDHSNLFIASSGVMPTAASVNCTLTIAALSLKLADKLKREI | 533 |

The present inventors studied the most suitable combination of mutations at positions 326, 365 and 472 by using genetic algorithm so as to increase the substrate specificity to glucose as compared to the mutant glucose dehydrogenase (CyGDH (QY)). As a result, the present inventors discovered a combination of mutations that can provide an improved substrate specificity.

A preferred mode of mutations of the mutant-type GDHs of the present invention is shown below.

(The numerals represent positions in the amino acid sequences; the amino acid residues represent amino acid residues after substitution at those positions; and, the symbol “+” means that two amino acids are simultaneously substituted.)

326Gln+365Tyr+472Tyr

The GDH α-subunits having the desired mutations can be obtained by introducing nucleotide mutations corresponding to the desired amino acids into a DNA coding for a GDH α-subunit (α-subunit gene) by site-specific mutagenesis, and expressing the obtained mutant DNA using an appropriate expression system. In addition, the mutant CyGDH complexes can be obtained by expressing a DNA coding for a mutant GDH α-subunit together with a DNA coding for a β-subunit (β-subunit gene) or together with a β-subunit gene and a DNA coding for a γ-subunit (γ-subunit gene). Alternatively, in introduction of mutations into a DNA coding for a GDH α-subunit, a polycistronic DNA fragment coding for a γ-subunit, an α-subunit and a β-subunit in the order mentioned may be used.

The substrate specificity of the GDH α-subunits or CyGDH complexes with the mutations introduced can be decided by examining their reactivities to various sugars with the methods as described in the Examples below and comparing those with the reactivities of wild-type GDH α-subunits or wild-type CyGDH complexes.

The polycistronic DNA fragments coding for a γ-subunit, an α-subunit and a β-subunit in the order mentioned can be obtained, for example, by PCR wherein a chromosomal DNA of Burkholderia cepacia KS 1 strain is used as a template and oligonucleotides having the nucleotide sequences of SEQ ID NOs:19 and 20 are used as primers (see the Examples as described below).

Examples of vectors used for obtaining these GDH subunit genes, introducing these mutations, and/or expressing these genes, etc., include, for example, a vector that functions in a bacterium belonging to genus Escherichia, more specifically pTrc99A, pBR322, pUC18, pUC118, pUC19, pUC119, pACYC184, pBBR122 and the like. Examples of promoters used for expressing these genes include, for example, lac, trp, tac, trc, P_{L}, tet, PhoA and the like. In addition, an α-subunit gene or other subunit gene can be inserted into an appropriate site of a promoter-containing expression vector, thereby accomplishing both of the insertion of the gene into the vector and the ligation with the promoter in one step. Examples of such expression vector include pTrc99A, pBluescript, pKK223-3 and the like.

Alternatively, the α-subunit gene or other subunit gene may be incorporated into a chromosomal DNA of a host microorganism in an expressible form.

Examples of methods for transforming microorganisms with recombinant vectors include, for example, a competent cell method by calcium treatment, a protoplast method, an electroporation method and the like.

Examples of the host microorganisms include bacteria belonging to genus Bacillus such as Bacillus subtilis, yeasts such as Saccharomyces cerevisiae, filamentous fungi such as Aspergillus niger. However, the host microorganisms which can be used are not limited to these examples, and other host microorganisms can be used as long as they are suitable to produce a foreign protein(s).

The mutant α-subunits or the mutant CyGDH complexes, or the microorganisms expressing those, of the present invention, can be used for an enzyme electrode of a glucose sensor or as a component of a glucose assay kit. A glucose sensor and a glucose assay kit which use a wild-type GDH of Burkholderia cepacia are described in U.S. Patent Publication No. 2004/0023330A1. The mutant GDHs of the present invention can be used in the same manner.

### EXAMPLES

The present invention will now be further described by way of Examples thereof. However, the present invention is not limited to these Examples in any way.

### Example 1

### Plasmids Expressing GDH or CyGDH of Burkholderia cepacia

As a plasmid expressing a GDH of Burkholderia cepacia, a plasmid expressing an α-subunit and a γ-subunit of the GDH was prepared; and, as a plasmid expressing a CyGDH, a plasmid expressing an α-subunit, a β-subunit and a γ-subunit was prepared.

### <1> Plasmid Expressing α-Subunit and β-Subunit of GDH

As a plasmid expressing an α-subunit and a γ-subunit, a plasmid pTrc99A/γ+α which is described in WO02/036779 (corresponding to EP1331272A1, US2004023330A1 and CN1484703A) was used. This plasmid is a plasmid wherein a DNA fragment contiguously containing a GDH γ-subunit structural gene and a GDH α-subunit structural gene, which was isolated from chromosomal DNA of Burkholderia cepacia KS1 strain (FERM BP-7306), is inserted into the NcoI/HindIII site which is a cloning site of the vector pTrc99A. The GDH γα gene in this plasmid is regulated by the trc promoter. The pTrc99A/γ+α has an ampicillin resistance gene.

The whole plasmid, including the DNA fragment which contains a sequence cording for six histidine residues added to the C-terminus of the GDH α-subunit, was amplified by PCR, wherein the above-mentioned plasmid pTrc99A/γ+α was used as a template and oligonucleotides having the following sequences were used as primers.

[Forward Primer]
(SEQ ID NO: 17)
5′-ACCACCACTGATAAGGAGGTCTGACCGTGCGGAAATCTAC-3′
[Reverse Primer]
(SEQ. ID NO: 18)
5′-AGCCTGTGCGACTTCTTCCTTCAGCGATCGGTGGTGGTGG-3′

Both termini of the amplified fragments were blunted. Thereafter, the 5′-terminal was phosphorylated, and the resulting fragments were circularized by ligation. Escherichia coli DH5α was transformed with the obtained recombinant vector, and colonies that were formed on LB agar medium containing 50 μg/mL of ampicillin were collected. The obtained transformants were cultured in liquid LB medium and the plasmid therein was extracted. The inserted DNA fragment was analyzed, and, as a result, about 2.1 kb of inserted fragment was confirmed. These GDH structural genes in this plasmid are regulated by the trc promoter. This plasmid has an ampicillin resistance gene.

### <2> Plasmid Expressing α-Subunit, β-Subunit and γ-Subunit of CyGDH

A plasmid expressing an α-subunit, a β-subunit and a γ-subunit of a CyGDH was prepared as described below.

(1) Preparing Chromosomal DNA from Burkholderia cepacia KS1 Strain

Chromosomal genes were prepared from Burkholderia cepacia KS1 strain according to a conventional method. In other words, cells of the strain were shaken in TL liquid medium (polypeptone 10 g, yeast extract 1 g, NaCl 5 g, KH₂PO₄ 2 g, glucose 5 g; 1 L, pH 7.2) at 34° C. overnight. The proliferated bacterial cells were collected by centrifugation. These bacterial cells were suspended in a solution containing 10 mM NaCl, 20 mM Tris-1-HCl (pH 8.0), 1 mM EDTA, 0.5% SDS, and 100 μg/ml of proteinase K and treated at 50° C. for 6 hours. An equal amount of phenol-chloroform was added to this solution and the resultant mixture was stirred at room temperature for 10 minutes. Thereafter, the supernatant was collected by centrifugation. Sodium acetate was added thereto so as to attain a final concentration of 0.3 M, and double the amount of ethanol was overlaid thereon to precipitate the chromosomal DNA in the middle layer. The precipitated DNA was spooled out using a glass rod, washed with 70% ethanol, and then dissolved in an appropriate amount of TE buffer to obtain a chromosomal DNA solution.

### (2) Preparing DNA Fragment Coding for γ-Subunit, α-Subunit and 13-Subunit of CyGDH

A DNA fragment coding for a γ-subunit, an α-subunit and a β-subunit of a CyGDH was amplified by PCR, wherein the above-mentioned chromosomal DNA was used as a template and oligonucleotides having the following sequences were used as primers.

[Forward Primer]
(SEQ ID NO: 19)
5′-CATGCCATGGCACACAACGACAACAC-3′
[Reverse Primer]
(SEQ ID NO: 20)
5′-GTCGACGATCTTCTTCCAGCCGAACATCAC-3′

The C-termini of the amplified fragments were blunted, and thereafter the N-termini were digested with NcoI. The resulting fragments were ligated to the pTrc99A treated in the same manner. Escherichia coli DH5α was transformed with the obtained recombinant vector, and colonies that were formed on LB agar medium containing 50 μg/mL of ampicillin were collected. The obtained transformants were cultured in liquid LB medium and the plasmid therein was extracted. The inserted DNA fragment was analyzed, and, as a result, about 3.8 kb of inserted fragments was confirmed. This plasmid was named pTrc99Aγαβ. These CyGDH structural genes in this plasmid are regulated by the trc promoter. The pTrc99Aγαβ has an ampicillin resistance gene and a kanamycin resistance gene.

### Example 2

### Search of Substrate Interaction Site by Introducing Mutations into CyGDH α-Subunit Gene

(1) Introducing Mutations into Positions 326, 365 and 472

Mutations were introduced into the GDH α-subunit gene contained in the pTrc99Aγαβ obtained in the Example 1 so that serine residue at position 326, serine residue at position 365 and alanine residue at position 472 of the α-subunit that was coded for by that gene were replaced with other amino acid residues.

Specifically, the codon corresponding to the serine at position 326 (TCG), the codon corresponding to the serine at position 365 (TCG) and the codon corresponding to the alanine at position 472 (GCG) of the GDH α-subunit genes contained in the plasmids pTrc99A/γ+α and pTrc99Aγαβ as described in Example 1 were replaced with codons corresponding to other amino acids using a commercially available site-specific mutagenesis kit (Stratagene, QuikChangeII Site-Directed Mutagenesis Kit).

The sequences of forward primers and reverse primers used in the above-mentioned amino acid residue substitutions are shown in Table 2 below. And, reverse primers used for introducing 3 mutations are shown in Table 3.

In the notations that represent the mutations, the numerals represent positions in the amino acid sequences, the alphabets before the numerals represent amino acid residues before the amino acid substitutions, and the alphabets after the numerals represent amino acid residues after the amino acid substitutions. For example, R53F represents that arginine at position 53 is replaced with phenylalanine.

PCR was carried out with the following reaction composition by performing a reaction of 95° C. for 30 seconds; then repeating 15 times a cycle of 95° C. for 30 seconds, 55° C. for 1 minute and 68° C. for 8 minutes; performing a reaction of 68° C. for 30 minutes; and then maintaining at 4° C.

### [Composition of the Reaction Solution]

| Template DNA (5 ng/μl) | 2 | μl |
|---|---|---|
| (pTrc99A/γ + α or pTrc99Aγαβ with | | |
| 3 mutations introduced) | | |
| 10× Reaction Buffer Solution | 5 | μl |
| Forward Primer (100 ng/μl) | 1.25 | μl |
| Reverse Primer (100 ng/μl) | 1.25 | μl |
| dNTPs | 1 | μl |
| Distilled Water | 38.5 | μl |
| DNA Polymerase | 1 | μl |
| Total | 50 | μl |

After the PCR, 0.5 μl of DpnI was added to the reaction solution, and the resultant mixture was incubated at 37° C. for 1 hour to degrade the template plasmid.

Competent cells of Escherichia coli DH5α (supE44, ΔlacU169 (φ80lacZΔM15), hsdR17, recAi, endA1, gyrA96, thi-1, relA1) were transformed using the obtained reaction solution. Each plasmid DNA was prepared from several colonies that had been grown on LB agar medium (bacto tryptone 1%, yeast extract 0.5%, sodium chloride 1% and agar 1.5%) containing ampicillin (50 μg/ml) and kanamycin (30 μg/ml), and the sequence thereof was analyzed to confirm that mutations of interest were introduced into the GDH α-subunit gene.

TABLE 2
Forward Primers for S326

| Amino acid | | | |
|---|---|---|---|
| substituion | Primer name | Sequence | SEQ ID NOs. |
| S326E | CGDH326ED | 5′GAATTCTATGCGAGCGAGAAGCTGTGGCCG3′ | SEQ ID NO: 21 |
| S326F | CGDH326FD | 5′TTCTTCTATGCGAGCGAGAAGCTGTGGCCG3′ | SEQ ID NO: 22 |
| S326I | CGDH326ID | 5′ATCTTCTATGCGAGCGAGAAGCTGTGGCCG3′ | SEQ ID NO: 23 |
| 5326K | CGDH326KD | 5′AAATTCTATGCGAGCGAGAAGCTGTGGCCG3′ | SEQ ID NO: 24 |
| S326L | CGDH326LD | 5′CTGTTCTATGCGAGCGAGAAGCTGTGGCCG3′ | SEQ ID NO: 25 |
| S326N | CGDH326ND | 5′AACTTCTATGCGAGCGAGAAGCTGTGGCCG3′ | SEQ ID NO: 26 |
| S326Q | CGDH326QD | 5′CAGTTCTATGCGAGCGAGAAGCTGTGGCCG3′ | SEQ ID NO: 27 |
| S326R | CGDH326RD | 5′CGCTTCTATGCGAGCGAGAAGCTGTGGCCG3′ | SEQ ID NO: 28 |
| S326T | CGDH326TD | 5′ACCTTCTATGCGAGCGAGAAGCTGTGGCCG3′ | SEQ ID NO: 29 |
| S326V | CGDH326VD | 5′GTTTTCTATGCGAGCGAGAAGCTGTGGCCG3′ | SEQ ID NO: 30 |
| S326W | CGDH326WD | 5′TGGTTCTATGCGAGCGAGAAGCTGTGGCCG3′ | SEQ ID NO: 31 |
| S326Y | CGDH326YD | 5′TACTTCTATGCGAGCGAGAAGCTGTGGCCG3′ | SEQ ID NO: 32 |

Reverse Primers for S326

| Primer name | Sequence | SEQ ID NOs. |
|---|---|---|
| CGDH326RSma | 5′CACGCCGGTCCCGGGATGGTCCATCAGGTT3′ | SEQ ID NO: 33 |
| CGDH326U | 5′CACGCCGGTGCCCGGAIGGTCCATCAGGTT3′ | SEQ ID NO: 34 |

Forward Primers for S365

| Amino acid | | | |
|---|---|---|---|
| substituion | Primer name | Sequence | SEQ ID NOs. |
| S365D | CGDH365DD | 5′GACAACCTGTCGCGCATCGACCAGGAGACG3′ | SEQ ID NO: 35 |
| 5365F | CGDH365FD | 5′TTCAACCTGTCGCGCATCGACCAGGAGACG3′ | SEQ ID NO: 36 |
| S365I | CGDH365ID | 5′ATCAACCTGTCGCGCATCGACCAGGAGACG3′ | SEQ ID NO: 37 |
| S365K | CGDH365KD | 5′AAAAACCTGTCGCGCATCGACCAGGAGACG3′ | SEQ ID NO: 38 |
| S365Q | CGDH365QD | 5′CAGAACCTGTCGCGCATCGACCAGGAGACG3′ | SEQ ID NO: 39 |
| S365R | CGDH365RD | 5′CGTAACCTGTCGCGCATCGACCAGGAGACG3′ | SEQ ID NO: 40 |
| S365T | CGDH365TD | 5′ACCAACCTGTCGCGCATCGACCAGGAGACG3′ | SEQ ID NO: 41 |
| S365Y | CGDH365YD | 5′TACAACCTGICGCGCATCGACCAGGAGACG3′ | SIE ID NO: 42 |

Reverse Primers for 5365

| Primer name | Sequence | SEQ ID NOs. |
|---|---|---|
| CGDH3651J | 5′CAGGTGGATCTTCTTCGCCGCTTCGGTCGC3′ | SEQ ID NO: 43 |

Forward Primers for A472

| Amino acid | | | |
|---|---|---|---|
| substituion | Primer name | Sequence | SEQ ID NOs. |
| A472A | CGDH472AD | 5′GCGCCGAACAATCACATCACGGGCTCGACG3′ | SEQ ID NO: 44 |
| A472D | CGDH472DD | 5′GACCCGAACAATCACATCACGGGCTCGACG3′ | SEQ ID NO: 45 |
| A472D | CGDH472ED | 5′GAACCGAACAATCACATCACGCGCTCGACG3′ | SEQ ID NO: 46 |
| A472F | CGDH472FD | 5′TTCCCGAACAATCACATCACGGGCTCGACG3′ | SEQ ID NO: 47 |
| A472H | CGDH472HD | 5′CATCCGAACAATCACATCACGGGCTCGACG3′ | SEQ ID NO: 48 |
| A472I | CGDH4721D | 5′ATCCCGAACAATCACATCACGGGCTCGACG3′ | SEQ ID NO: 49 |
| A472K | CGDH472KD | 5′AAACCGAACAATCACATCACGGCCTCGACG3′ | SEQ ID NO: 50 |
| A472N | CGDH472ND | 5′AACCCGAACAATCACATCACGGGCTCGACG3′ | SEQ ID NO: 51 |
| A472R | CGDH472RD | 5′CGTCCGAACAATCACATCACGGGCTCGACG3′ | SEQ ID NO: 52 |
| A472S | CGDH472SD | 5′AGTCCGAACAATCACATCACGGGCTCGACG3′ | SEQ ID NO: 53 |
| A472T | CGDH472TD | 5′ACTCCGAACAATCACATCACGGGCTCGACG3′ | SEQ ID NO: 54 |
| A472Y | CGDH472YD | 5′TACCCGAACAATCACATCACGGGCTCGACG3′ | SEQ ID NO: 55 |

Reverse Primers for A472

| Primer name | Sequence | SEQ ID NOs. |
|---|---|---|
| CGDH472U | 5′GAATFCGTCGTTGAACACGACGTCCGTGCC3′ | SEQ ID NO: 56 |
| CGDH472UdEI | 5′GAACTCGICGTTGAACACGACGICCGTGCC3′ | SEQ ID NO: 57 |

TABLE 3
List of Three Mutations and Reverse Primer Used

| | Reverse primer | Reverse primer | Reverse primer |
|---|---|---|---|
| | for S326 | for S365 | for A472 |
| TTS | CGDH326RSma | CGDH365U | CGDH472UdE1 |
| TDF | CGDH326U | CGDH365U | CGDH472UdE1 |
| YKY | CGDH326U | CGDH365U | CGDH472UdE1 |
| YQE | CGDH326U | CGDH365U | CGDH472U |
| QII | CGDH326RSma | CGDH365U | CGDH472U |
| QDN | CGDH326RSma | CGDH365U | CGDH472U |
| ERY | CGDH326RSma | CGDH365U | CGDH472UdE1 |
| EFE | CGDH326RSma | CGDH365U | CGDH472UdE1 |
| IKD | CGDH326U | CGDH365U | CGDH472UdE1 |
| QYR | CGDH326U | CGDH365U | CGDH472U |
| LTN | CGDH326U | CGDH365U | CGDH472U |
| RYK | CGDH326U | CGDH365U | CGDH472U |
| KYT | CGDH326RSma | CGDH365U | CGDH472U |
| RRI | CGDH326U | CGDH365U | CGDH472U |
| KYI | CGDH326RSma | CGDH365U | CGDH472U |
| YYS | CGDH326U | CGDH365U | CGDH472U |
| IFK | CGDH326U | CGDH365U | CGDH472UdE1 |
| IFR | CGDH326U | CGDH365IJ | CGDH472UdE1 |
| FRY | CGDH326U | CGDH365U | CGDH472U |
| FRA | CGDH326U | CGDH365U | CGDH472U |
| EYT | CGDH326RSma | CGDH365U | CGDH472UdE1 |
| FRE | CGDH326U | CGDH365U | CGDH472U |
| RKA | CGDH326RSma | CGDH365U | CGDH472U |
| RRY | CGDH326U | CGDH365U | CGDH472U |
| KYR | CGDH326U | CGDH365U | CGDH472U |
| KYS | CGDH326RSma | CGDH365U | CGDH472UdE1 |
| WFK | CGDH326U | CGDH365U | CGDH472UdE1 |
| IRY | CGDH326U | CGDH365U | CGDH472U |
| IYT | CGDH326U | CGDH365U | CGDH472U |
| IYH | CGDH326U | CGDH365U | CGDH472U |
| EFK | CGDH326RSma | CGDH365U | CGDH472UdE1 |
| FRH | CGDH326U | CGDH365U | CGDH472U |
| NRF | CGDH326U | CGDH365U | CGDH472UdE1 |
| NRT | CGDH326U | CGDH365U | CGDH472UdE1 |
| VRK | CGDH326U | CGDH365U | CGDH472UdE1 |
| KYH | CGDH326RSma | CGDH365U | CGDH472UdE1 |
| LYF | CGDH326RSma | CGDH365U | CGDH472U |
| QRY | CGDH326U | CGDH365U | CGDH472U |
| QYY | CGDH326U | CGDH365U | CGDH472U |
| KRH | CGDH326RSma | CGDH365U | CGDH472U |

### Example 3

### Analysis of Substrate Specificities of Mutant GDHs

Mutant GDHs were produced by using the plasmids expressing mutant GDHs which were obtained in the Example 2, and their substrate specificities were studied.

### (1) Culture

The cells of Escherichia coli DH5α strain wherein mutations were introduced in various patterns were each cultured under shaking at 37° C. overnight in 2 ml of LB medium (containing 50 μg/ml of ampicillin and 30 μg/ml of kanamycin) by using a L-shaped tube. These cultures were inoculated into a 500 ml Sakaguchi flask that contained 150 ml of LB medium (containing 50 μg/ml of ampicillin and 30 μg/ml of kanamycin), and the resultants were cultured under shaking at 37° C. Three hours after the beginning of the culturing, IPTG (isopropyl-β-D-thiogalactopyranoside) was added so as to attain a final concentration of 0.1 mM, and the resultants were cultured for another 2 hours.

### (2) Preparing Crude Enzyme Samples

The bacterial cells were collected from the cultures which had been cultured as described above, and the collected cells were washed. Thereafter, the bacterial cells were suspended with 1 ml of 10 mM potassium phosphate buffer (PPB) (pH 7.0) containing 0.2% Triton X100 per 0.3 mg of wet bacterial cells, and sonicated. These suspensions were centrifuged (10,000 r.p.m, 10 minutes, 4° C.) to remove the residues. Thereafter, the supernatants were ultracentrifuged (50,000 r.p.m., 60 minutes, 4° C.), and the obtained supernatants (water-soluble fractions) were considered as crude enzyme samples. Further, these samples were purified by usual hydrophobic chromatography (column name: Octyl Sepharose (manufactured by Amersham Biosciences) and ion exchange chromatography (Q-Sepharose (manufactured by Amersham Biosciences) to obtain purified enzyme samples. Which fractions contained the enzymes of interest was decided by using their GDH activity as an index.

Furthermore, purification of His-tag containing proteins was performed as described below.

The bacterial cells were collected from the cultures which had been cultured, and the collected cells were washed. Thereafter, the bacterial cells were suspended with 1 ml of 20 mM sodium phosphate buffer (pH 7.5) containing 0.5 M sodium chloride and 20 mM imidazole per 0.3 mg of wet bacterial cells, and sonicated. These suspensions were centrifuged (10,000 r.p.m, 10 minutes, 4° C.) to remove the residues. Thereafter, the supernatants were ultracentrifuged (50,000 r.p.m., 60 minutes, 4° C.), and the obtained supernatants (water-soluble fractions) were considered as crude enzyme samples. These samples were added into HisTrap FF columns (manufactured by Amersham Biosciences) equilibrated with 20 mM sodium phosphate buffer (pH 7.5) containing 0.5 M sodium chloride and 20 mM imidazole. The columns were washed with 20 mM sodium phosphate buffer (pH 7.5) containing 0.5 M sodium chloride and 60 mM imidazole, then subjected to elution with 20 mM sodium phosphate buffer (pH 7.5) containing 0.5 M sodium chloride and 150 mM imidazole to obtain purified enzyme samples. Which fractions contained the enzymes of interest was decided by using their GDH activity as an index,

### (3) Measurement of GDH Activity

To 8 μl of each crude enzyme sample as mentioned above, 8 μl of a reagent for measuring the activity (a solution that was obtained by adding 10 mM PPB containing 0.2 (w/v) % Triton X-100 to 12 μl of 600 mM methylphenazine methosulfate (PMS) and 120 μl of 6 mM 2,6-dichlorophenolindophenol (DCIP) so as to attain a total volume of 480 μl) was added. The obtained mixture was preincubated at each reaction temperature for 1 minute by using an aluminum block thermostat, and then 8 μl of each concentration of substrate (glucose or maltose) or distilled water was quickly added. The resultant solution was stirred, and absorbance at 600 nm, which is an absorption wavelength of DCIP, was measured by using a spectrophotometer. The final concentrations of the reagents DCIP and PMS were 0.06 mM and 0.6 mM, respectively. The final concentrations of the substrates were 10 mM or 5 mM.

The results are shown in Table 4. At 10 mM and 5 mM of substrate concentrations, the reaction ratios of the wild-type GDH were 23.32% and 18.88%, respectively.

TABLE 4

| U/mg | 10 mM | | | | | |
|---|---|---|---|---|---|---|
| crude | | | Mal/ | 5 mM | | |
| protein | Glu | Mal | Glu (%) | Glu | Mal | Mal/Glu (%) |
| WT | 4.69 | 1.09 | 23.32 | 3.99 | 0.75 | 18.88 |
| QYA | 0.68 | 0.0065 | 0.96 | 0.43 | 0.0038 | 0.88 |
| TTS | 1.40 | 0.23 | 16.18 | 1.22 | 0.19 | 15.25 |
| TDF | 0.37 | 0.17 | 45.16 | 0.23 | 0.12 | 52.63 |
| YKY | 0.14 | 0.07 | 51.72 | 0.10 | 0.05 | 50.00 |
| YQE | 0.06 | 0.01 | 9.52 | 0.04 | 0.00 | 8.46 |
| QII | 0.40 | 0.06 | 15.00 | 0.24 | 0.03 | 13.89 |
| QDN | 0.17 | 0.08 | 47.42 | 0.11 | 0.05 | 48.44 |
| ERY | 0.29 | 0.002 | 0.79 | 0.17 | 0.001 | 0.78 |
| EFE | 0.05 | 0.001 | 2.74 | 0.03 | 0.001 | 2.75 |
| IKD | 0.01 | 0.029 | 236.36 | 0.01 | 0.019 | 188.89 |
| QYR | 1.90 | 0.0180 | 0.97 | 1.21 | 0.0112 | 0.93 |
| LTN | 0.07 | 0.0140 | 18.95 | 0.05 | 0.0105 | 19.44 |
| RYK | 1.08 | 0.2400 | 22.09 | 0.75 | 0.1348 | 17.90 |
| KYT | 1.135 | 0.0082 | 0.721 | 0.669 | 0.0048 | 0.714 |
| RRI | 0.08 | 0.0048 | 6.32 | 0.06 | 0.0037 | 5.70 |
| KYI | 0.12 | 0.0022 | 1.80 | 0.07 | 0.0018 | 2.53 |
| YYS | nd | nd | nd | nd | nd | nd |
| IFK | 1.27 | 0.0380 | 2.99 | 0.81 | 0.0243 | 3.00 |
| IFR | 0.28 | 0.0094 | 3.38 | 0.18 | 0.0060 | 3.36 |
| FRY | 0.34 | 0.0060 | 1.76 | 0.26 | 0.0037 | 1.42 |
| FRA | 0.14 | 0.0086 | 6.09 | 0.13 | 0.0065 | 5.08 |
| YET | 0.13 | 0.0026 | 1.94 | 0.09 | 0.0024 | 2.79 |
| FRE | nd | nd | nd | nd | nd | nd |
| RKA | nd | nd | nd | nd | nd | nd |
| RRY | 0.49 | 0.0050 | 1.01 | 0.38 | 0.0034 | 0.88 |
| KYR | 1.67 | 1.61E−02 | 0.97 | 1.09 | 8.68E−03 | 0.80 |
| KYS | 0.47 | 3.64E−03 | 0.78 | 0.29 | 1.82E−03 | 0.63 |
| WFK | 0.61 | 2.47E−02 | 4.03 | 0.43 | 1.38E−02 | 3.22 |
| IRY | 0.24 | 5.01E−02 | 21.2 | 0.18 | 4.75E−02 | 26.54 |
| IYT | 0.21 | — | — | 0.13 | — | — |
| IYH | 0.16 | — | — | 0.09 | — | — |
| EFK | 0.18 | 5.74E−03 | 3.20 | 0.10 | 3.23E−03 | 3.21 |
| FRH | 0.05 | — | — | 0.47 | — | — |
| NRF | 0.19 | — | — | 0.13 | — | — |
| NRT | 0.26 | 1.13E−02 | 4.31 | 0.23 | 5.85E−03 | 2.55 |
| VRK | 0.09 | 4.74E−03 | 5.42 | 0.08 | 2.55E−03 | 3.18 |
| KYH | 0.04 | — | — | 0.02 | — | — |
| LYF | 0.15 | — | — | 0.09 | — | — |
| QRY | 0.35 | 7.64E−02 | 21.8 | 0.26 | 0.0531 | 20.5 |
| QYY | 0.55 | 1.91E−03 | 0.35 | 0.31 | 0.00143 | 0.46 |
| KRH | nd | nd | nd | nd | nd | nd |

In this Table, QYA represents a mutant glucose dehydrogenase whose amino acid residues corresponding to positions 326 and 365 are replaced with glutamine and tyrosine, respectively, but whose amino acid residue corresponding to position 472 is not replaced (that is to say, the amino acid residue corresponding to position 472 remains to be an alanine residue), i.e., a GDH (QY).

As a result, it was found that 326Glu+365Arg+472Tyr, 326Gln+365Tyr+472Arg, 326Lys+365Tyr+472Thr, 326IIe+365Phe+472Lys, 326Arg+365Arg+472Tyr, 326Lys+365Tyr+472Arg, 326Lys+365Tyr+472Ser, and 326Gln+365Tyr+472Tyr were very effective mutations wherein the reactivities to glucose were maintained and the reactivities to maltose were reduced.

In other words, a synergistic effect equal to or more than that of the mutant GDH (QY) was confirmed in some of the combinations of the three mutations at positions 326, 365 and 472.

### Example 4

### Preparing Purified Enzymes (Complexes of α-Subunit and γ-Subunit)

The 8 mutant GDHs which showed improved substrate specificities in Example 3 were purified. The same method as described in Example 3 was used. Specific activities to glucose and maltose (U/mg), reaction ratios (specific activity to maltose/specific activity to glucose), and Km values and Vmax values for glucose, of each purified enzyme, are shown in Table 5.

As a result, it was found that the reaction ratios of QYA with 10 mM and 5 mM of maltose were both at the level of 1%, whereas the reaction ratios of ERY and KYT were reduced to 1% or less and the reaction ratios of QYY with maltose were reduced to the level of 0.3%.

TABLE 5

| | 10 mM | | | | | | | | | |
|---|---|---|---|---|---|---|---|---|---|---|
| | Glu | | | 5 mM | | | Glucose | | | Mal/Glu |
| | (U/mg) | Mal (U/mg) | Mal/Glu (%) | Glu (U/mg) | Mal (U/mg) | Mal/Glu (%) | Km (mM) | Vmax (U/mg) | Vmax/Km | (%) |
| WT his | 90.2 | 23.7 | 26.3 | 77.6 | 15.8 | 20.4 | 1.35 | 100 | 74.1 | 10.9 |
| QYA his | 55.3 | 0.56 | 1.02 | 32.8 | 0.41 | 1.25 | 4.32 | 62.9 | 14.6 | 2.08 |
| RYT his | 5.18 | 0.05 | 1.03 | 2.89 | 0.03 | 1.05 | 27.5 | 19.1 | 0.69 | 1.32 |
| KYK his | 5.27 | 0.09 | 1.67 | 3.51 | 0.05 | 1.54 | 10.5 | 10.9 | 1.04 | 1.37 |
| ERY his | 9.53 | 0.08 | 0.89 | 6.07 | 0.05 | 0.75 | 22.9 | 33.4 | 1.46 | 2.26 |
| QYR his | 41.3 | 0.44 | 1.07 | 25.1 | 0.37 | 1.47 | 5.34 | 55.6 | 10.4 | 1.90 |
| KYT his | 33.3 | 0.21 | 0.63 | 19.6 | 0.16 | 0.81 | 8.13 | 52.1 | 6.41 | 3.26 |
| IFK his | 5.08 | 0.23 | 4.49 | 3.39 | 0.13 | 3.85 | 6.52 | 7.70 | 1.18 | 2.90 |
| RRY his | 2.64 | 0.04 | 1.67 | 2.15 | 0.03 | 1.36 | 3.82 | 3.82 | 1.00 | 2.40 |
| QYY his | 32.2 | 0.09 | 0.29 | 20.2 | 0.07 | 0.34 | 13.8 | 76.3 | 5.53 | unmeasurable |

DCIP 0.6 mM; PMS 6 mM, room temperature

### Example 5

### Preparing Purified Enzymes (Complexes of α-Subunit, γ-Subunit and γ-Subunit)

The mutant CyGDH (QYY) which showed an improved substrate specificity in Example 3 was purified. The same method as described in Example 3 was used. Specific activities to glucose (Wing) of each purified enzyme are shown in Table 6.

As a result, it was found that, in the form of a complex of an α-subunit, a β-subunit and a γ-subunit, the reactivity with maltose of QYY (326Gln 365Tyr+472Tyr) were reduced to about ½ to ⅓ as compared to that of QYA (326Gln+365Tyr).

TABLE 6

| | 0.1 | 0.5 | 1 | 2 | 5 | 7 | 10 | 15 | 20 |
|---|---|---|---|---|---|---|---|---|---|
| Glucose (mM) | | | | | | | | | |
| QYA (U/mg protein) | 1.37 | 24.7 | 60.4 | 131 | 250 | 350 | 420 | 513 | 551 |
| QYY (U/mg protein) | 0.80 | 9.66 | 35.4 | 62.7 | 139 | 202 | 233 | 324 | 409 |
| WT (U/mg protein) | 5.28 | 45.2 | 108 | 263 | 421 | 485 | 584 | 631 | 651 |
| Maltose | | | | | | | | | |
| QYA (U/mg protein) | 0.009 | 0.047 | 0.17 | 0.14 | 0.66 | 0.66 | 1.38 | 3.13 | 3.44 |
| QYY (U/mg protein) | 0.005 | 0.015 | 0.03 | 0.07 | 0.11 | 0.31 | 0.45 | 0.59 | 0.99 |
| WT (U/mg protein) | 1.41 | 5.41 | 13.9 | 23.5 | 42.7 | 48.7 | 65.6 | 80.7 | 83.4 |
| Mal/Glu | | | | | | | | | |
| QYA | 0.69% | 0.19% | 0.28% | 0.11% | 0.27% | 0.19% | 0.33% | 0.61% | 0.62% |
| QYY | 0.62% | 0.16% | 0.08% | 0.12% | 0.08% | 0.16% | 0.19% | 0.18% | 0.24% |
| WT | 26.7% | 12.0% | 12.9% | 8.95% | 10.1% | 10.0% | 11.2% | 12.8% | 12.8% |

### Example 6

### Analysis of Substrate Specificities of Mutant Enzymes (QYX Mutation)

Mutations were introduced in the same manner as in Example 2 into the GDH α-subunit gene contained in the pTrc99A/γ+α so that serine residue at position 326, serine residue at position 365 and alanine residue at position 472 of the α-subunit that was coded for by that gene were replaced with glutamine, tyrosine and an amino acid residue other than alanine, respectively. Using the obtained plasmids expressing mutant enzymes, in the same manner as in Example 3, mutant enzymes were produced, and their substrate specificities were studied. The results are shown in Table 7 below.

TABLE 7

| | Glucose | | | Maltose | | | | | |
|---|---|---|---|---|---|---|---|---|---|
| | | | 5 mM/ | | | 5 mM/ | 5 mM Mal/ | 50 mM Mal/ | 50 mM Mal/ |
| U/mL sample | 5 mM | 50 mM | 50 mM | 5 mM | 50 mM | 50 mM | 5 mM Glc | 50 mM Glc | 5 mM Glc |
| QYA (Wild) | 5.68 | 14.60 | 38.9% | 0.07 | 0.36 | 18.2% | 1.1% | 2.5% | 6.3% |
| QYC | 0.46 | 0.90 | 51.7% | 0.00 | 0.05 | 0.0% | 0.0% | 5.6% | 10.9% |
| QYD | 3.31 | 12.69 | 26.1% | 0.08 | 0.28 | 27.2% | 2.3% | 2.2% | 8.4% |
| QYE | 2.44 | 11.96 | 20.4% | 0.00 | 0.16 | 0.0% | 0.0% | 1.4% | 6.7% |
| QYF | 0.46 | 8.48 | 5.5% | 0.01 | 0.05 | 16.1% | 1.8% | 0.6% | 11.0% |
| QYG | 3.27 | 9.58 | 34.2% | 0.05 | 0.12 | 44.7% | 1.7% | 1.3% | 3.7% |
| QYH | 0.85 | 6.30 | 13.5% | 0.00 | 0.07 | 0.0% | 0.0% | 1.1% | 8.3% |
| QYI | 2.64 | 10.72 | 24.7% | 0.00 | 0.18 | 0.0% | 0.0% | 1.7% | 6.8% |
| QYK | 3.06 | 9.08 | 33.8% | 0.03 | 0.34 | 7.7% | 0.9% | 3.8% | 11.2% |
| QYL | 7.48 | 20.86 | 35.8% | 0.04 | 0.27 | 16.5% | 0.6% | 1.3% | 3.6% |
| QYM | 2.10 | 11.42 | 18.4% | 0.01 | 0.29 | 1.8% | 0.3% | 2.5% | 13.8% |
| QYN | 1.88 | 14.77 | 12.7% | 0.05 | 0.23 | 22.3% | 2.7% | 1.6% | 12.3% |
| QYP | 1.88 | 7.47 | 25.2% | 0.01 | 0.06 | 24.7% | 0.7% | 0.7% | 3.0% |
| QYQ | 4.75 | 15.41 | 30.8% | 0.06 | 0.34 | 17.6% | 1.3% | 2.2% | 7.2% |
| QYR | 4.30 | 12.58 | 34.2% | 0.06 | 0.33 | 19.8% | 1.5% | 2.6% | 7.6% |
| QYS | 2.19 | 12.00 | 18.2% | 0.00 | 0.18 | 0.0% | 0.0% | 1.5% | 8.1% |
| QYT | 3.23 | 12.54 | 25.8% | 0.06 | 0.20 | 32.5% | 2.0% | 1.6% | 6.1% |
| QYV | 5.11 | 15.91 | 32.1% | 0.05 | 0.31 | 15.0% | 0.9% | 1.9% | 6.0% |
| QYW | 0.80 | 9.35 | 8.6% | 0.00 | 0.11 | 0.0% | 0.0% | 1.2% | 13.5% |
| QYY | 2.38 | 14.49 | 16.4% | 0.01 | 0.09 | 8.9% | 0.3% | 0.6% | 3.6% |

As a result, it was found that QYD, QYG, QYK, QYL, QYP, QYQ, QYR, QYT, QYV and QYY were mutations wherein the reactivities to glucose were maintained and the reactivities with maltose were reduced.

### Example 7

### Preparing Purified Enzymes (Complexes of α-Subunit and β-Subunit)

The 10 mutant GDHs which showed improved substrate specificities in Example 6 (QYD, QYG, QYK, QYL, QYP, QYQ, QYR, QYT, QYV and QYY) were purified. The same method as described in Example 3 was used. Specific activities to glucose and maltose (U/mg), reaction ratios (specific activity to maltose/specific activity to glucose), and Km values and Vmax values for glucose, of each purified enzyme, are shown in Table 8.

As a result, it was found that the reaction ratios of the mutant GDHs with maltose were all low, but, except for QYY, the enzyme activities of these mutant GDHs were reduced, suggesting that these mutant GDHs except for QYY are not suitable for measuring the glucose concentration. In other words, it is suggested that, among the 10 mutant GDHs represented by QYX, QYY is most suitable for measuring the glucose concentration.

TABLE 8

| QYY | | | | | | | |
|---|---|---|---|---|---|---|---|
| Km (Glc.) = 30.1 mM | | | | | | | |
| Vmax (Glc.) = 131.6 U/mg protein | | | | | | | |
| U/mg | 5 mM | 10 mM | 20 mM | | | | |
| Glucose | 21.89 | 28.69 | 52.39 | | | | |
| | (100.0%) | (131.0%) | (239.3%) | | | | |
| Maltose | 0.06 | 0.13 | 0.29 | | | | |
| | (0.3%) | (0.6%) | (1.3%) | | | | |
| QYA | | | | QYR | | | |
| Km (Glc.) = 16.1 mM | | | | Km (Glc.) = 11.2 mM | | | |
| Vmax (Glc.) = 149.3 U/mg protein | | | | Vmax (Glc.) = 93.5 U/mg protein | | | |
| U/mg | 5 mM | 10 mM | 20 mM | U/mg | 5 mM | 10 mM | 20 mM |
| Glucose | 34.91 | 56.61 | 83.94 | Glucose | 30.00 | 42.68 | 59.73 |
| | (100.0%) | (162.1%) | (240.5%) | | (100.0%) | (142.3%) | (199.1%) |
| Maltose | 0.40 | 0.64 | 1.45 | Maltose | 0.32 | 0.59 | 1.09 |
| | (1.1%) | (1.8%) | (4.1%) | | (1.1%) | (2.0%) | (3.6%) |
| QYD | | | | QYP | | | |
| Km (Glc.) = 24.9 mM | | | | Km (Glc.) = 35.5 mM | | | |
| Vmax (Glc.) = 64.1 U/mg protein | | | | Vmax (Glc.) = 74.1 U/mg protein | | | |
| U/mg | 5 mM | 10 mM | 20 mM | U/mg | 5 mM | 10 mM | 20 mM |
| Glucose | 10.26 | 18.78 | 29.32 | Glucose | 9.53 | 16.78 | 25.23 |
| | (100.0%) | (183.1%) | (285.8%) | | (100.0%) | (176.0%) | (264.8%) |
| Maltose | 0.02 | 0.18 | 0.31 | Maltose | 0.09 | 0.13 | 0.15 |
| | (0.2%) | (1.7%) | (3.0%) | | (0.9%) | (1.4%) | (1.6%) |
| QYG | | | | QYQ | | | |
| Km (Glc.) = 15.7 mM | | | | Km (Glc.) = 15.7 mM | | | |
| Vmax (Glc.) = 59.2 U/mg protein | | | | Vmax (Glc.) = 65.4 U/mg protein | | | |
| U/mg | 5 mM | 10 mM | 20 mM | U/mg | 5 mM | 10 mM | 20 mM |
| Glucose | 12.92 | 23.54 | 31.56 | Glucose | 15.36 | 24.94 | 38.55 |
| | (100.0%) | (182.2%) | (244.3%) | | (100.0%) | (162.4%) | (251.0%) |
| Maltose | 0.18 | 0.25 | 0.38 | Maltose | 0.21 | 0.42 | 1.03 |
| | (1.4%) | (1.9%) | (3.0%) | | (1.4%) | (2.7%) | (6.7%) |
| QYK | | | | QYT | | | |
| Km (Glc.) = 10.8 mM | | | | Km (Glc.) = 36.3 mM | | | |
| Vmax (Glc.) = 78.7 U/mg protein | | | | Vmax (Glc.) = 88.5 U/mg protein | | | |
| U/mg | 5 mM | 10 mM | 20 mM | U/mg | 5 mM | 10 mM | 20 mM |
| Glucose | 28.02 | 33.68 | 52.64 | Glucose | 9.68 | 20.44 | 32.87 |
| | (100.0%) | (120.2%) | (187.8%) | | (100.0%) | (211.3%) | (339.7%) |
| Maltose | 0.48 | 0.65 | 1.23 | Maltose | 0.14 | 0.21 | 0.33 |
| | (1.7%) | (2.3%) | (4.4%) | | (1.5%) | (2.2%) | (3.4%) |
| QYL | | | | QYV | | | |
| Km (Glc.) = 13.4 mM | | | | Km (Glc.) = 18.3 mM | | | |
| Vmax (Glc.) = 94.3 U/mg protein | | | | Vmax (Glc.) = 108.7 U/mg protein | | | |
| U/mg | 5 mM | 10 mM | 20 mM | U/mg | 5 mM | 10 mM | 20 mM |
| Glucose | 23.95 | 37.28 | 57.03 | Glucose | 27.25 | 35.53 | 54.04 |
| | (100.0%) | (155.7%) | (238.1%) | | (100.0%) | (130.4%) | (198.3%) |
| Maltose | 0.23 | 0.34 | 0.66 | Maltose | 0.15 | 0.32 | 0.80 |
| | (1.0%) | (1.4%) | (2.8%) | | (0.6%) | (1.2%) | (2.9%) |

(DCIP f.c. 0.06 mM; PMS f.c 6 mM, 10 mM PPB pH 7.0)

### Example 8

### Production of Colorimetric Sensor for Measuring Blood Sugar Level Using Mutant CyGDH

With respect to the QYY, which had the best specific activity as described above, a glucose sensor was produced using the purified mutant enzyme obtained in the Example 5.

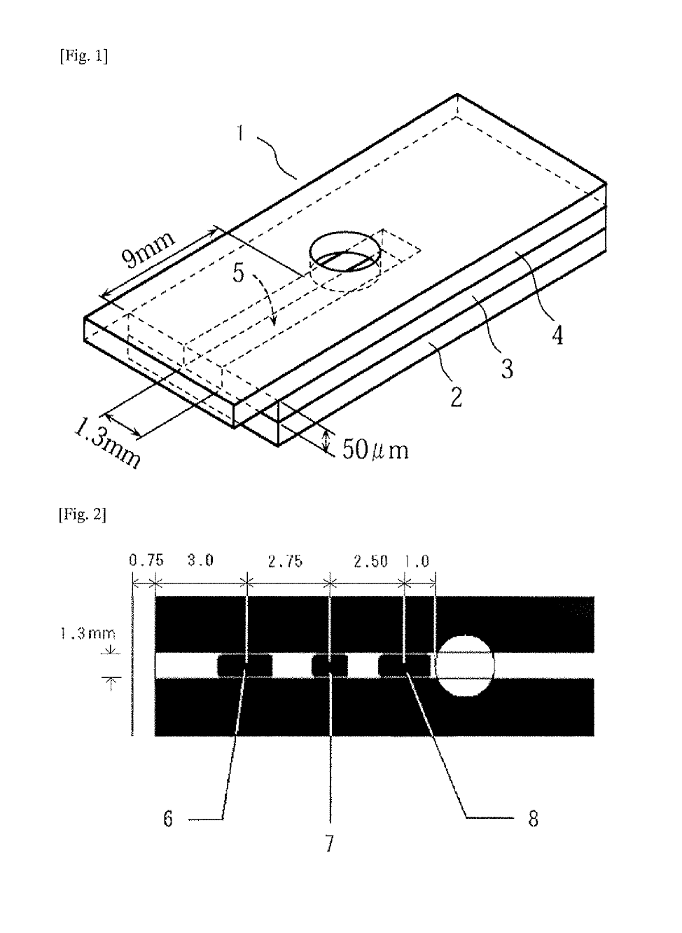

A glucose sensor having the basic structure as shown in FIG. 1 was produced. That is to say, this glucose sensor has a configuration wherein the transparent cover 4 (material: PET) is laminated on the transparent base plate 2 via the spacer 3, and the capillary 5 is defined by these elements 2 to 4. The dimension of the capillary 5 is 1.3 mm×9 mm×50 μm (FIG. 1). The transparent base plate 2 and the transparent cover 4 were formed with PET having a thickness of 250 μm, and the spacer 3 was formed with a black double-sided tape.

The glucose sensor has first to third reagent parts as shown in FIG. 2. Ingredients and coating amounts for each reagent part are shown in Table 9. In this Table, “Ru” represents a ruthenium hexaammine complex (Ru(NH₃)₆Cl₃); ACES represents N-(2-acetamido)-2-aminoethanesulfonic acid; WST-4 represents 2-Benzothiazolyl-3-(4-carboxy-2-methoxyphenyl)-5-[4-(2-sulfoethylcarbamoyl)phenyl]-2H-tetrazolium; and SWN represents Lithium Magnesium Sodium Silicate.

TABLE 9
First reagent part
Material solution for reagent part containing electron
transfer substance (solvent is water)

| Ru | Coating amount |
|---|---|
| 60 mM | 0.5 μl |

Second reagent part
Material solution for reagent part
containing enzyme (solvent is water)

| Enzyme | | Sucrose | ACES | Coating |
|---|---|---|---|---|
| concentration | Raffinose | monolaurate | (pH 7.5) | amount |
| 12 KU/ml | 1.0% | 0.1% | 100 mM | 0.2 μl |

Third reagent part
Material solution for reagent part containing
color developer (solvent is water)

| WST-4 | SWN | Coating amount |
|---|---|---|
| About 50 mM | 0.16% | 0.4 μl |

Each sample to be measured was supplied to the capillary of the glucose sensor as mentioned above, and, at 5 seconds after that, absorbance of the end point was plotted. In these absorbance measurements, the third reagent part was irradiated with light along the height direction of the capillary, and, at that time, the light transmitted through the glucose sensor was received. The light irradiation was performed by irradiating with 630 nm light using a light emitting diode. The transmitted light was received with a photodiode.

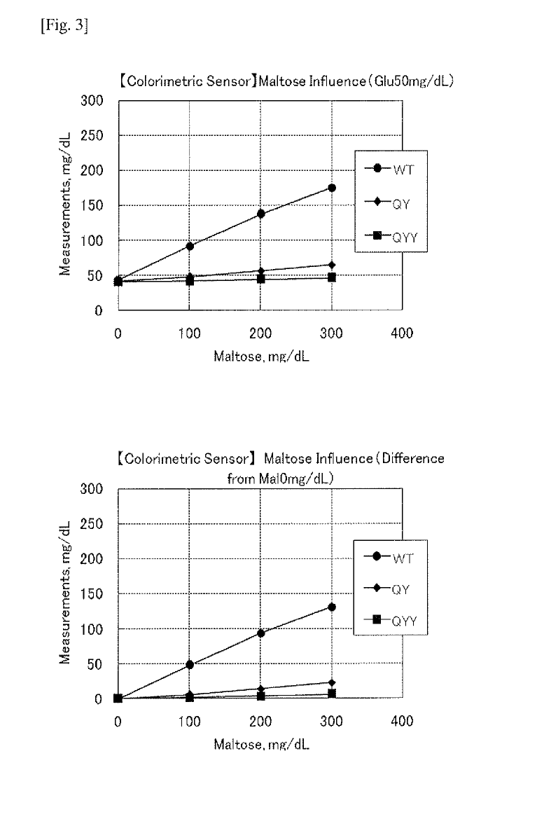

As for the influence of maltose, when maltose is added to 50 mg/dl of glucose, the absorbance in the case of the wild type is largely increased depending on the maltose concentration, which suggests that the wild type reacts strongly with maltose. On the other hand, with respect to the sensor that uses the mutant CyGDH (QY), it is found that the absorbance increase depending on the maltose concentration is suppressed, and thus the influence of maltose is reduced. With respect to the sensor that uses the mutant CyGDH (QYY), it is found that the absorbance increase depending on the maltose concentration is further suppressed, and thus the influence of maltose is further reduced. These data were converted into apparent blood sugar elevation values, and the results are shown in FIG. 3.

In the sensor that uses the wild-type enzyme, a low blood sugar level (50 mg/dl of glucose) is apparently indicated as a level above the normal range (174.8 mg/dl of glucose) due to the presence of maltose (ratio of change: 300%). Even in the sensor that uses the modified CyGDH (QY), the low blood sugar level is also apparently indicated as 64.8 mg/dl of glucose due to the presence of maltose (ratio of change: 54%). On the other hand, in the sensor that uses the modified CyGDH (QYY), even if maltose is present in an amount up to 300 mg/dl, the apparent blood sugar level is elevated only up to at most 46.1 mg/dl, and therefore it can be said that the influence is significantly suppressed (ratio of change: 14%). In conclusion, it is suggested that the QYY mutant is most suitable from the point of view of the reactivity to glucose (linearity) and influence of maltose.

As is clear from the above results, in the glucose sensor that uses the mutant CyGDH (QYY), the reactivity with maltose is largely reduced as compared to that in the glucose sensor that uses the mutant CyGDH (QY). If this glucose sensor that uses the mutant CyGDH (QYY) is used, then it can be said that there will be no difference between right and wrong at 200 mg/dl of maltose, which is the upper limit of blood maltose level that can be administered in a hospital or the like; and, even at 300 mg/dl of maltose, which is above the upper limit, low blood sugar levels (50 mg/dl or less) are not judged as normal levels or high blood sugar levels, thereby allowing for safe therapeutic treatments.

### Example 9

### Production of Electrode Sensor for Measuring Blood Sugar Level Using Mutant CyGDH

With respect to the QYY, which had the best specific activity as described above, an electrode glucose sensor was produced using the purified mutant enzyme obtained in the Example 5.

### (Procedure for Producing)

The method of producing an electrode glucose sensor is shown below with reference to FIG. 5. A base plate made of PET (28 mm in length, 7 mm in width, and 250 μm in thickness) was prepared as the insulating base plate 1, and, on one surface thereof, a carbon electrode system consisting of the working electrode 4 and the counter electrode 5, which had the lead parts 2 and 3, respectively, was formed by screen printing of carbon ink. Then, an insulating paste was prepared and screen-printed on the above-mentioned electrodes to form the insulating layer 6. By this screen printing, the parts wherein the insulating layer was not formed were obtained as the detecting part and the lead part, respectively. Then, 0.6 g of “Lucentite SWN” which is a trade name of a synthetic smectite (manufactured by Co-op Chemical Co., Ltd.) was suspended in 100 mL of purified water, and the resultant suspension was stirred for about 8 to 24 hours. To 10 mL of this suspension of the synthetic smectite, 0.1 mL of 10% (w/v) aqueous solution of CHAPS (manufactured by DOJINDO LABORATORIES), 5.0 mL of 1.0 M ACES buffer solution (pH 7,4, manufactured by DOJINDO LABORATORIES) and 4.0 mL of purified water were added in the order mentioned. Further, the resultant solution was mixed with 1.0 g of [Ru(NH₃)₆]Cl₃ (manufactured by Aldrich) as a mediator. To the detecting part, 1.0 μL of this mixture was dispensed, and the dispensed mixture was dried under the conditions of 30° C. and 10% of relative humidity for 10 minutes to form the reagent layer 7. Further, the enzyme reagent layer 8 was formed on this reagent layer 7. This enzyme reagent layer 8 was formed by dispensing 1.0 μL of 2700 U/mL aqueous solution of the glucose dehydrogenase of the present invention onto the reagent layer of the detecting part, and drying the dispensed solution under the conditions of 30° C. and 10% of relative humidity for 10 minutes. Finally, the spacer 11 having the slit 15 was placed on the insulating layer, and the cover 12 having a penetrating pore, i.e., the air hole 14, was further placed on this spacer to produce a biosensor.

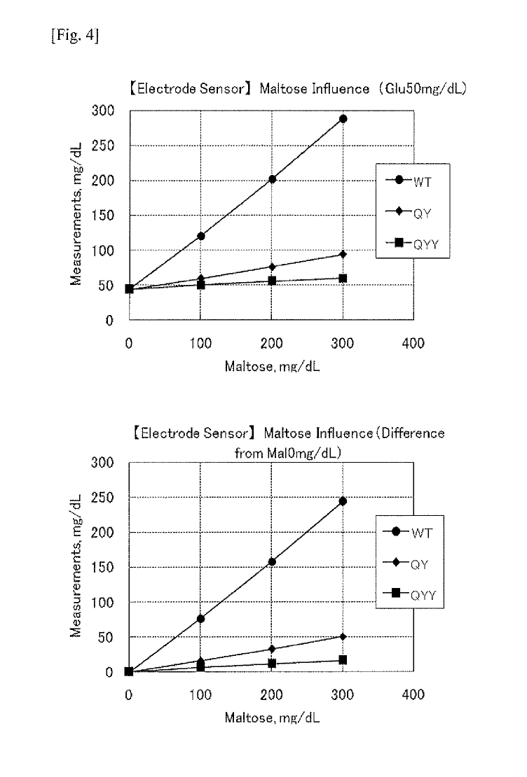

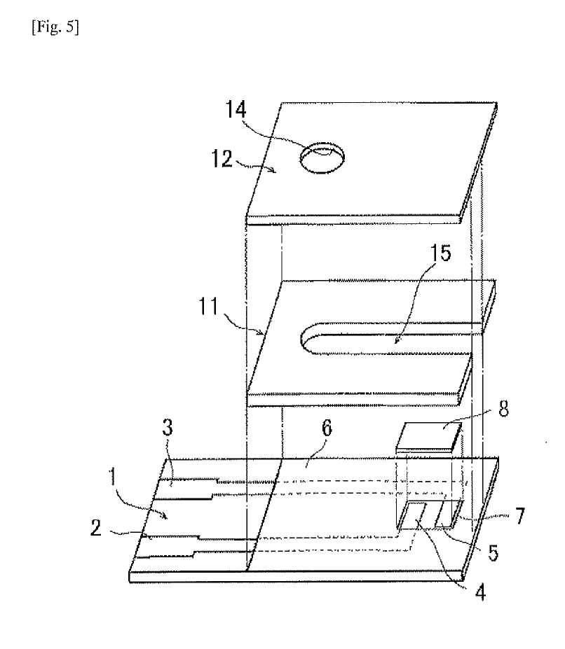

As for the influence of maltose, when maltose is added to 50 mg/dl of glucose, the electric current value in the case of the wild type is largely increased depending on the maltose concentration, which suggests that the wild type reacts strongly with maltose. On the other hand, with respect to the sensor that uses the mutant CyGDH (QY), it is found that the electric current value increase depending on the maltose concentration is suppressed, and thus the influence of maltose is reduced. With respect to the sensor that uses the mutant CyGDH (QYY), it is found that the electric current value increase depending on the maltose concentration is further suppressed, and thus the influence of maltose is further reduced. These data were converted into apparent blood sugar elevation values, and the results are shown in FIG. 4.

In the sensor that uses the wild-type enzyme, a low blood sugar level (50 mg/dl of glucose) is apparently indicated as a level above the normal range (288.2 mg/dl of glucose) due to the presence of maltose (ratio of change: 549%). Even in the sensor that uses the modified CyGDH (QY), the low blood sugar level is also apparently indicated as 93.9 mg/dl of glucose due to the presence of maltose (ratio of change: 114%). On the other hand, in the sensor that uses the modified CyGDH (QYY), even if maltose is present in an amount up to 300 mg/dl, the apparent blood sugar level is elevated only up to at most 60.0 mg/dl, and therefore it can be said that the influence is significantly suppressed (ratio of change: 37%). In conclusion, it is suggested that the QYY mutant is most suitable from the point of view of the reactivity to glucose (linearity) and influence of maltose.

As is clear from the above results, in the glucose sensor that uses the mutant CyGDH (QYY), the reactivity with maltose is largely reduced as compared to that in the glucose sensor that uses the mutant CyGDH (QY). If this glucose sensor that uses the mutant CyGDH (QYY) is used, then it can be said that there will be no difference between right and wrong at 200 mg/dl of maltose, which is the upper limit of blood maltose level that can be administered in a hospital or the like; and, even at 300 mg/dl of maltose, which is above the upper limit, low blood sugar levels (50 mg/dl or less) are not judged as normal levels or high blood sugar levels, thereby allowing for safe therapeutic treatments.

### Example 10

### Introducing Mutations into GDH Homologues

Plasmids expressing each α- and γ-subunits of a putative oxidoreductase of Burkholderia cenocepacia J2315 strain, a hypothetical protein BthaT_{—}07876 of Burkholderia thailandensis TXDOH strain, a FAD dependent oxidoreductase of Ralstonia pickettii 12D strain, a transmembrane dehydrogenase of Ralstonia solanacearum IPO1609 strain and a glucose-methanol-choline oxidoreductase of Burkholderia phytofirmans PsJN strain were prepared in the same manner as in Example 1.

These expression plasmids are plasmids wherein a DNA fragment contiguously containing a γ-subunit structural gene and an α-subunit structural gene is inserted into the NdeI/HindIII site, which is a cloning site of the vector pET30c(+) (Novagen). The γα gene in this plasmid is regulated by the T7 promoter. This plasmid has a kanamycin resistance gene.

Using a commercially available site-specific mutagenesis kit (Stratagene, QuikChangeII Site-Directed Mutagenesis Kit), mutations were introduced into the α-subunit gene contained in the above-mentioned plasmids so that residues corresponding to positions 326, 365 and 472 of the wild-type GDH α-subunit of Burkholderia cepacia KS1 strain were replaced with glutamine, tyrosine and tyrosine, respectively.

The sequences of forward primers used for these amino acid residue substitutions are shown below. The sequences of reverse primers were strands fully complementary to these forward primers.

In the notations that represent the mutations, the numerals represent positions in the amino acid sequences, the alphabets before the numerals represent amino acid residues before the amino acid substitutions, and the alphabets after the numerals represent amino acid residues after the amino acid substitutions. For example, R53F represents that arginine at position 53 is replaced with phenylalanine.

PCR was carried out with the following reaction composition by performing a reaction of 95° C. for 30 seconds; then repeating 15 times a cycle of 95° C. for 30 seconds, 55° C. for 1 minute and 68° C. for 8 minutes; performing a reaction of 68° C. for 30 minutes; and then maintaining at 4° C.

### [Composition of the Reaction Solution]

| | | |
|---|---|---|
| Template DNA (5 ng/μl) | 2 | μl |
| (pTrc99A/γ + α or pTrc99Aγαβ with | | |
| 3 mutations introduced) | | |
| 10× Reaction Buffer Solution | 5 | μl |
| Forward Primer (100 ng/μl) | 1.25 | μl |
| Reverse Primer (100 ng/μl) | 1.25 | μl |
| dNTPs | 1 | μl |
| Distilled Water | 38.5 | μl |
| DNA Polymerase | 1 | μl |
| Total | 50 | μl |

After the PCR, 0.5 μl of DpnI was added to the reaction solution, and the resultant mixture was incubated at 37° C. for 1 hour to degrade the template plasmid.

Competent cells of Escherichia coli DH5α(supE44, ΔlacU169 (φ80lacZΔM15), hsdR17, recAi, endA1, gyrA96, thi-1, relA1) were transformed using the obtained reaction solution. Each plasmid DNA was prepared from several colonies that had been grown on LB agar medium (bacto tryptone 1%, yeast extract 0.5%, sodium chloride 1% and agar 1.5%) containing ampicillin (50 μg/ml) and kanamycin (30 μg/ml), and the sequence thereof was analyzed to confirm that mutations of interest were introduced into each α-subunit gene.

TABLE 10
Forward Primers for Introducing Mutations into Putative Oxidoreductase of
Burkholderia cenocepacia J2315 Strain

| Amino acid | | |
|---|---|---|
| substituion | Sequence | SEQ ID NOs. |
| S326Q | 5′GGGCACCGGCGTGCAGTTCTATGCGAACGAG 3′ | SEQ ID NO: 58 |
| S365Y | 5′GAAGAAGATCCACCTGTACAACATGTCGCGCATCAAC 3′ | SEQ ID NO: 59 |
| A472Y | 5′GTCGTGTTCAACGACGAATTCTACCCGAACAATCACATC | SEQ ID NO: 60 |
| | ACGGG 3′ | |

Reverse Primers for Introducing Mutations into Putative Oxidoreductase of
Burkholderia cenocepacia J2315 Strain

| Amino acid | | |
|---|---|---|
| substituion | Sequence | SEQ ID NOs. |
| S326Q | 5′CTCGTTCGCATAGAACTGCACGCCGGTGCCC 3′ | SEQ ID NO: 61 |
| S365Y | 5′GTTGATGCGCGACATGTTGTACAGGTGGATCTTCTTC 3′ | SEQ ID NO: 62 |
| A472Y | 5′CCCCGTGATGTGATTGTTCGGGTAGAATTCGTCGTTGAAC | SEQ ID NO: 63 |
| | ACGAC 3′ | |

Forward Primers for Introducing Mutations into Hypothetical Protein
BthaT_07876 of Burkholderia thailandensis TXDOH Strain

| Amino acid | | |
|---|---|---|
| substituion | Sequence | SEQ ID NOs. |
| S324Q | 5′GGGCACCGGCGTGCAGTTCTATGCGAGCGAG 3′ | SEQ ID NO: 64 |
| S363Y | 5′GAAGAAGATCCACCTGTACAACCTGTCGCGCATCGAC 3′ | SEQ ID NO: 65 |
| A470Y | 5′GTCGTGTTCAACGACGAATTCTACCCGAACAATCACATC | SEQ ID NO: 60 |
| | ACGGG 3′ | |

Reverse Primers for Introducing Mutations into Hypothetical Protein
BthaT_07876 of Burkholderia thailandensis TXDOH Strain

| Amino acid | | |
|---|---|---|
| substituion | Sequence | SEQ ID NOs. |
| S324Q | 5′CTCGCTCGCATAGAACTGCACGCCGGTGCCC 3′ | SEQ ID NO: 66 |
| S363Y | 5′GTCGATGCGCGACAGGTTGTACAGGTGGATCTTCTTC 3′ | SEQ ID NO: 67 |
| A470Y | 5′CCCGTGATGTGATTGTTCGGGTAGAATTCGTCGTTGAAC | SEQ ID NO: 63 |
| | ACGAC 3′ | |

Forward Primers for Introducing Mutations into FAD Dependent Oxidoreductase
of Ralstonia pickettii 12D Strain

| Amino acid | | |
|---|---|---|
| substituion | Sequence | SEQ ID NOs. |
| S327Q | 5′GGGCACCGGCGTGCAGTTCTATGCGGACCGC 3′ | SEQ ID NO: 68 |
| S366Y | 5′CAAGAAGCTGCACCGTACAACATCTCGCGCATCGAG 3′ | SEQ ID NO: 69 |
| A473Y | 5′GTCGAGTTCCACGACGACTTCTACCCGAACAATCACATC | SEQ ID NO: 70 |
| | ACGGG 3′ | |

Reverse Primers for Introducing Mutations into FAD Dependent Oxidoreductase
of Ralstonia pickettii 12D Strain

| Amino acid | | |
|---|---|---|
| substituion | Sequence | SEQ ID NOs. |
| S327Q | 5′GCGGtCCGCATAGAACTGCACGCCGGTGCCC 3′ | SEQ ID NO: 71 |
| S366Y | 5′CTCGATGCGCGAGATGTTGTACAGGTGCAGCTTCTtG 3′ | SEQ ID NO: 72 |
| A473Y | 5′CCCGTGATGTGATTGTTCGGGTAGAAGTCGTCGTGGAAC | SEQ ID NO: 73 |
| | TCGAC 3′ | |

Forward Primers for Introducing Mutations into Transmembrane Dehydrogenase
of Ralstonia solancicearum IPO1609 Strain

| Amino acid | | |
|---|---|---|
| substituion | Sequence | SEQ ID NOs. |
| S327Q | 5′GGGCACCGGCGTGCAGTTCTATGCGGACCCC 3′ | SEQ ID NO: 68 |
| S366Y | 5′CAAGAAGCTGCACCTGTACAACATCTCGCGCATCGAG 3′ | SEQ ID NO: 69 |
| A473Y | 5′GTCCAGTTCCACGACGACTTCTACCCGAACAATCACATC | SEQ ID NO: 74 |
| | ACGGG 3′ | |

Reverse Primers for Introducing Mutations into Transmembrane Dehydrogenase
of Ralstonia solanacearum IPO1609 Strain

| Amino acid | | |
|---|---|---|
| substituion | Sequence | SEQ ID NOs. |
| S327Q | 5′GCGGTCCGCATAGAACTGCACGCCOGTGCCC 3′ | SEQ ID NO: 71 |
| S366Y | 5′CTCGATGCGCGAGATGTTGTACAGOTGCAGCTTCTTG 3′ | SEQ ID NO: 72 |
| A473Y | 5′CCCGTGATGTGATTGTTCGGGTAGAAGTCGTCGTGGAAC | SEQ ID NO: 75 |
| | TGGAC 3′ | |

Forward Primers for Introducing Mutations into Glucose-Methanol-Choline
Oxidoreductase of Burkholderia phytofirmans PsJN Strain

| Amino acid | | |
|---|---|---|
| substituion | Sequence | SEQ ID NOs. |
| S322Q | 5′GGGCACCGGCGTGCAGTTCCTGGCGAACGAG 3′ | SEQ ID NO: 76 |
| S361Y | 5′GAAGAAGCTGCACCTGTACAACGGCGTCCCGACGATG 3′ | SEQ ID NO: 77 |
| A466Y | 5′GTCACGTTCGACGACACGTTCTACCCGAACAATCACATC | SEQ ID NO: 78 |
| | ATGGG 3′ | |

Reverse Primers for Introducing Mutations into Glucose-Methanol-Choline
Oxidoreductase of Burkholderia phytofirmans PsJN Strain

| Amino acid | | |
|---|---|---|
| substituion | Sequence | SEQ ID NOs. |
| S322Q | 5′CTCGTTCGCCAGGAACTGCACGCCGGTGCCC 3′ | SEQ ID NO: 79 |
| 5361Y | 5′CATCGTCGGGACGCCGTTGTACAGGTGCAGCTTCTTC 3′ | SEQ ID NO: 80 |
| A466Y | 5′CCCATGATGTGATTGTTCGGGTAGAACGTGTCGTCGAAC | SEQ ID NO: 81 |
| | GTGAC 3′ | |

### Example 11

### Analysis of Substrate Specificities of Mutant Enzymes

Mutant enzymes were produced by using the plasmids expressing mutant enzymes which were obtained in the Example 10, and their substrate specificities were studied.

### (1) Culture

Competent cells of Escherichia coli BL21 (DE3)(F—, dcm, ompT, hsdS (rB- mB-), gal, λ (DE3)) were transformed with each of the plasmids expressing mutant enzymes. The obtained transformants were each cultured under shaking at 37° C. overnight in 3 ml of LB medium (containing 25 μg/ml of kanamycin) by using a L-shaped tube. These cultures were inoculated into a 500 ml Sakaguchi flask that contained 100 ml of LB medium (containing 0.5% glycerol, 0.05% glucose, 0.2% lactose, 100 mM PO₄, 25 mM SO₄, 50 mM NH₄, 100 mM Na, 50 mM K, 1 mM MgSO₄ and 25 μg/ml of kanamycin), and the resultants were cultured under shaking at 20° C. for 24 hours.

### (2) Preparing Crude Enzyme Samples

The bacterial cells were collected from the cultures which had been cultured as described above, and the collected cells were washed. Thereafter, the bacterial cells were suspended with 1 ml of 10 mM potassium phosphate buffer (PPB) (pH 7.0) per 0.1 g of wet bacterial cells, and sonicated. These suspensions were centrifuged (10000 r.p.m, 10 minutes, 4° C.) to remove the residues. Thereafter, the supernatants were ultracentrifuged (50,000 r.p.m., 60 minutes, 4° C.), and the obtained supernatants (water-soluble fractions) were considered as crude enzyme samples.

### (3) Measurement of GDH Activity

To 10 μl of each crude enzyme sample as mentioned above, 170 μl of a reagent for measuring the activity (a solution that was obtained by adding 10 mM PPB to 94 μl of 600 mM methylphenazine methosulfate (PMS) and 9.4 μl of 6 mM 2,6-dichlorophenolindophenol (DCIP) so as to attain a total volume of 8 ml) was added. To the obtained mixture, 20 μl of each concentration of substrate (glucose or maltose) or distilled water was added. The resultant solution was stirred, and absorbance at 600 nm, which is an absorption wavelength of DCIP, was measured by using a spectrophotometer. The final concentrations of the reagents DCIP and PMS were 0.06 mM and 0.6 mM, respectively.

The results are shown in Table 11. In all of these mutant enzymes, it was confirmed that the reactivities to maltose were reduced and the substrate specificities to glucose were improved as compared to the wild-type enzymes before the mutation introduction.

TABLE 11

| | | 10 mM | | | 5 mM | | | Glc | | | Mal | | |
|---|---|---|---|---|---|---|---|---|---|---|---|---|---|
| | | Glc | Mal | | Glc | Mal | | Km | Vmax | | Km | Vmax | Vmax/ |
| | | (U/mL) | (U/mL) | Mal/Glc | (U/mL) | (U/mL) | Mal/Glc | (mM) | (U/mL) | Vmax/Km | (mM) | (U/mL) | Km |
| B. conocopacia J2315 | WT | 2.02 | 1.64 | 80.8% | 2.02 | 1.48 | 72.9% | 0.11 | 2.05 | 18.6 | 1.3 | 1.87 | 1.44 |
| putative oxidoreductase | QYY | 11.4 | 0.31 | 2.7% | 7.59 | 0.15 | 2.0% | 20.3 | 39.2 | 1.93 | >100 | — | — |
| B. thailandensis TXDOH | WT | 20.1 | 12.0 | 59.8% | 19.6 | 9.58 | 49.0% | 0.31 | 20.7 | 86.8 | 2.67 | 15.5 | 5.81 |
| hypothetical protein | QYY | 20.7 | 0.22 | 1.1% | 13.6 | 0.13 | 1.0% | 13.0 | 48.8 | 3.76 | >100 | — | — |
| BthaT_07876 | | | | | | | | | | | | | |
| R. pickott [symbol] 12D | WT | 0.94 | 0.78 | 82.5% | 0.89 | 0.68 | 76.6% | 0.36 | 1.03 | 2.86 | 2.02 | 0.94 | 0.47 |
| FAD dependent | QYY | 0.73 | 0.03 | 3.7% | 0.50 | 0.02 | 3.0% | 11.0 | 1.62 | 0.16 | >100 | — | — |
| oxidoreductase | | | | | | | | | | | | | |
| R. solanacoar [symbol] m IPO1609 | WT | 0.13 | 0.11 | 83.6% | 0.12 | 0.09 | 74.7% | 0.59 | 0.14 | 0.24 | 2.64 | 0.14 | 0.05 |
| transmembrane | QYY | 0.58 | 0.01 | 2.4% | 0.40 | 0.01 | 2.2% | 13.9 | 1.55 | 0.11 | >100 | — | — |
| dehydrogenase | | | | | | | | | | | | | |

[symbol]

indicates data missing or illegible when filed

From the results as described above, it is suggested that the QYY mutation is also useful for GDH homologues of strains other than Burkholderia cepacia.

### INDUSTRIAL APPLICABILITY

The mutant GDHs of the present invention show improved substrate specificities to glucose, and can be suitably used for measuring glucose using a glucose sensor or the like.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

1. A mutant glucose dehydrogenase having an amino acid sequence at least 80% identical to SEQ ID NO:3 and having glucose dehydrogenase activity,
wherein amino acid residues corresponding to positions 326, 365 and 472 of said amino acid sequence are replaced with glutamine. tyrosine and tyrosine, respectively, and
wherein said mutant glucose dehydrogenase shows an improved substrate specificity to glucose and a reduced reactivity to disaccharides.

2. The mutant glucose dehydrogenase according to claim 1, which has an amino acid sequence at least 90% identical to SEQ 10 NO:3.

3. The mutant glucose dehydrogenase according to claim 1, which has the amino acid sequence of SEQ ID NO:3 except for the positions corresponding to positions 326, 365 and 472.

4. The mutant glucose dehydrogenase according to claim 1, which has the amino acid sequence of SEQ ID NO:7 except for the positions corresponding to positions 326, 365 and 472.

5. The mutant glucose dehydrogenase according to claim 1. which has the amino acid sequence of SEQ ID NO:8 except for the positions corresponding to positions 326, 365 and 472.

6. The mutant glucose dehydrogenase according to claim 1, which has the amino acid sequence of SEQ ID NO:9 except for the positions corresponding to positions 326, 365 and 472.

7. The mutant glucose dehydrogenase according to claim 1, which has the amino acid sequence of SEQ ID NO: I 0 except for the positions corresponding to positions 326, 365 and 472.

8. The mutant glucose dehydrogenase according to claim 1, wherein said mutant glucose dehydrogenase shows an improved substrate specificity to glucose and a reduced reactivity to disaccharides as compared to a glucose dehydrogenase whose amino acid residues corresponding to positions 326 and 365 in said amino acid sequence are replaced with glutamine and tyrosine, respectively, but whose amino acid residue corresponding to position 472 is not replaced.

9. The mutant glucose dehydrogenase according to claim 1. wherein the disaccharides are maltose.

10. A mutant glucose dehydrogenase complex comprising at least the mutant glucose dehydrogenase according to claim 1 and an electron transfer subunit.

11. The glucose dehydrogenase complex according to claim 10, wherein the electron transfer subunit is cytochrome C.

12. A DNA coding for the mutant glucose dehydrogenase according to claim 1.

13. A microorganism having the DNA according to claim 12 and producing the mutant glucose dehydrogenase.

14. A microorganism having the DNA according to claim 12 and producing a mutant glucose dehydrogenase complex comprising at least the mutant glucose dehydrogenase and an electron transfer subunit.

15. A glucose assay kit comprising the mutant glucose dehydrogenase according to claim 1.

16. A glucose assay kit comprising the mutant glucose dehydrogenase complex according to claim 10.

17. A glucose assay kit comprising the microorganism according to claim 13.

18. A glucose sensor comprising the mutant glucose dehydrogenase according to claim 1.

19. A glucose sensor comprising the mutant glucose dehydrogenase complex according 10 claim.

20. A glucose sensor comprising the microorganism according to claim 13.
